(19) United States
(12) Patent Application Publication
(10) Pub. No.: US 2023/0131192 A1
(43) Pub. Date: Apr. 27, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Christine M. TARBY, Lawrenceville, NJ (US); Matthias BROEKEMA, New Hope, PA (US); Ashvinikumar V. GAVAI, Princeton Junction, NJ (US); Sanjeev GANGWAR, Foster City, CA (US); Naidu S. CHOWDARI, Dublin, CA (US); Walter L JOHNSON, San Francisco, CA (US); Murugaiah ANDAPPAN MURUGAIAH SUBBAIAH, Bommasandra Bangalore (IN)

(21) Appl. No.: 17/793,155

(22) PCT Filed: Jan. 26, 2021
(86) PCT No.: PCT/US2021/014978

§ 371 Date: Jul. 15, 2022

Related U.S. Application Data
(60) Provisional application No. 63/058,130, filed on Jul. 29, 2020.
(60) Provisional application No. 62/966,124, filed on Jan. 27, 2020.

(51) Int. Cl. C07D 487/04; A61K 45/06

(52) U.S. Cl. CPC C07D 487/04; A61K 45/06

(57) ABSTRACT

Compounds according to formula I are useful as agonists of Toll-like receptor 7 (TLR7). (I) Such compounds can be used in cancer treatment, especially in combination with an anti-cancer immunotherapy agent, or as a vaccine adjuvant.

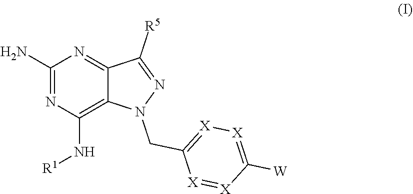

### CROSS-REFERENCE TO RELATED APPLICATIONS

This application claims the benefit under 35 U.S.C. § 119(e) of U.S. Provisional Application Ser. No. 63/058,130, filed Jul. 29, 2020, and U.S. Provisional Application Ser. No. 62/966,124, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

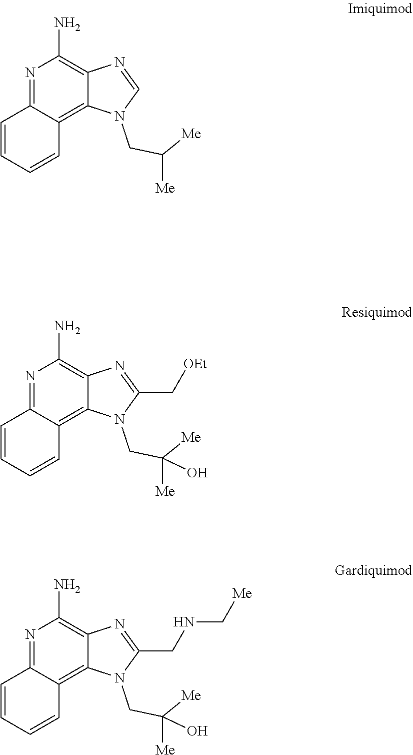 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

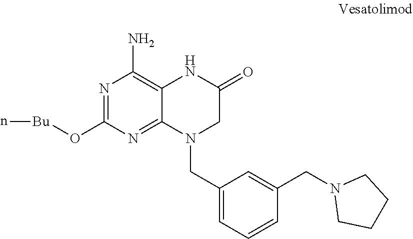

Other synthetic TLR7 agonists based on a purine-like scaffold have been disclosed, frequently according to the general formula (A):

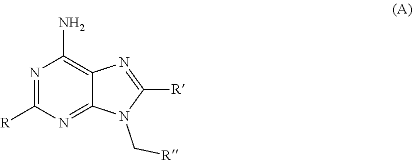

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

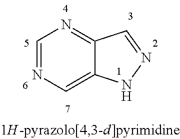

In one aspect, there is provided a compound with a structure according to formula (I)

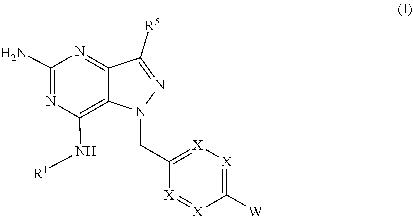

wherein
W is H, halo, C₁-C₃ alkyl, CN, (C₁-C₄ alkanediyl)OH,

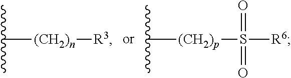

each X is independently N or CR²;
R¹ is (C₁-C₈ alkanediyl)₀₋₁(C₃ cycloalkyl),
   (C₁-C₈ alkanediyl)₀₋₁(C₅-C₆ cycloalkyl),
   (C₁-C₄ alkanediyl)₀₋₁(5-6 membered heteroaryl),
   (C₁-C₄ alkanediyl)₀₋₁phenyl, or
   (C₁-C₄ alkanediyl)CF₃;
each R² is independently H, O(C₁-C₃ alkyl), S(C₁-C₃ alkyl), SO₂(C₁-C₃ alkyl), C₁-C₃ alkyl,
   O(C₃-C₄ cycloalkyl), S(C₃-C₄ cycloalkyl), SO₂(C₃-C₄ cycloalkyl), C₃-C₄ cycloalkyl, Cl, F, CN; or
   [C(═O)]₀₋₁NR^{x}R^{y};
R³ is H, halo, OH, CN,
   NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₅ cycloalkyl),
   N(C₃-C₆ cycloalkyl)₂,
   N[C₁-C₃ alkyl]C(═O)(C₁-C₆ alkyl),
   NH(SO₂)(C₁-C₅ alkyl),
   NH(SO₂)(C₁-C₄ alkanediyl)₀₋₁(C₃-C₅ cycloalkyl),
   a 6-membered aromatic or heteroaromatic moiety,
   a 5-membered heteroaromatic moiety, or
   a moiety having the structure

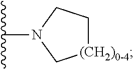

R⁵ is H, C₁-C₅ alkyl, C₂-C₅ alkenyl, C₃-C₆ cycloalkyl, halo, O(C₁-C₅ alkyl),
   (C₁-C₄ alkanediyl)OH, (C₁-C₄ alkanediyl)O(C₁-C₃ alkyl), phenyl, NH(C₁-C₅ alkyl), 5 or 6 membered heteroaryl,

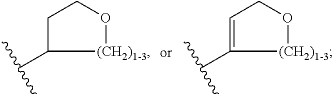

R⁶ is NH₂,
   (NH)₀₋₁(C₁-C₅ alkyl),
   N(C₁-C₅ alkyl)₂,
   (NH)₀₋₁(C₁-C₄ alkanediyl)₀₋₁(C₃-C₈ cycloalkyl),
   N(C₃-C₆ cycloalkyl)₂,
   or
   a moiety having the structure

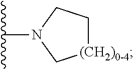

R^{x} and R^{y} are independently H or C₁-C₃ alkyl or R^{x} and R^{y} combine with the nitrogen to which they are bonded to form a 3- to 7-membered ring
n is 1, 2, or 3;
and
p is 0, 1, 2, or 3;
wherein in R1, R², R³, and R⁵
   an alkyl moiety, alkanediyl moiety, cycloalkyl moiety, or moiety of the formula

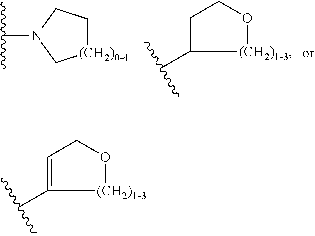

is optionally substituted with one or more substituents selected from OH, halo, CN,
(C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or moiety of the formula

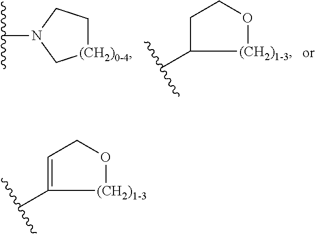

may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, compounds of this disclosure are according to formula (Ia), wherein R1 and R³ are as defined in respect of formula (I):

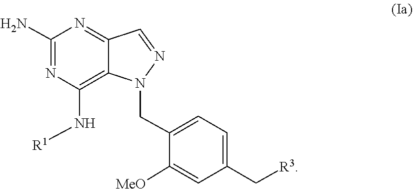

In one aspect, this disclosure provides a compound having a structure according to formula (Ia) wherein

### R¹ is

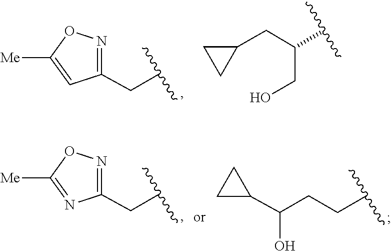 [SMILES: *Cc1cc(C)on1 ; *[C@H](CO)CC1CC1 ; *Cc1noc(C)n1 ; *CCC(O)C1CC1]

and

### R³ is OH,

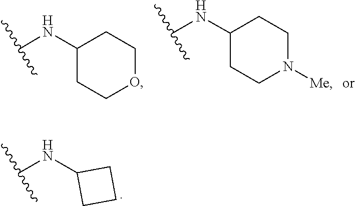

Examples of groups R1 include:

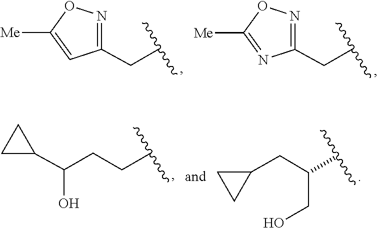 [SMILES: *Cc1cc(C)on1 ; *Cc1noc(C)n1 ; *CCC(O)C1CC1 ; *[C@H](CO)CC1CC1]

R² preferably is OMe, O(cyclopropyl), or OCHF₂, more preferably OMe.

Examples of groups R³ include OH

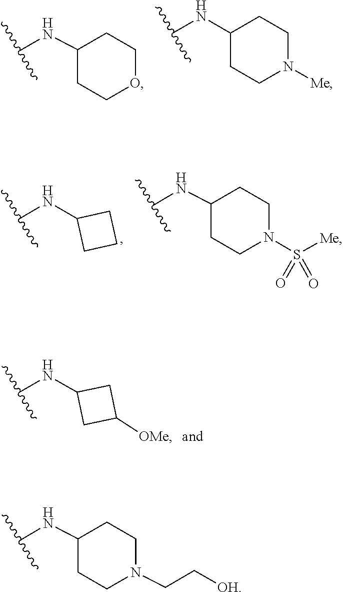

In one aspect, R⁵ is H.

Specific examples of compounds disclosed herein are shown in the following Table A. The table also provides data relating to biological activity: human TLR7 reporter assay and/or induction of the CD69 gene in human whole blood, determined per the procedure provided hereinbelow. The right-most column contains analytical data (mass spectrum, HPLC retention time, and NMR). In one embodiment, a compound of this disclosure has (a) a human TLR7 (hTLR7) agonist (Reporter) Assay EC₅₀ value of less than 1,000 nM and (b) a human whole blood (hWB) CD69 induction EC₅₀ value of less than 1,000 nM. (Where an assay was performed multiple times, the reported value is an average.)

TABLE A

| | | hTLR7 | hWB | Analytical Data (Mass spectrum, LC/MS |
|---|---|---|---|---|
| Cpd | | Agonism | CD69 | Retention Time, ¹H NMR (500 MHz, |
| No. | Structure | EC₅₀ (nM) | EC₅₀ (nM) | DMSO-d6 unless noted otherwise)) |
| 101 | 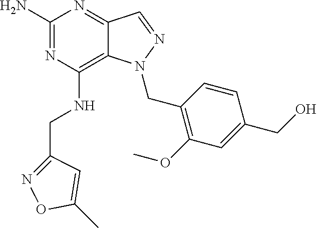 [SMILES: COc1cc(CO)ccc1Cn1ncc2nc(N)nc(NCc3cc(C)on3)c21] | 354.0 | 471.6 | LC/MS [M + H]⁺ = 396.3 RT (min) = 1.09 (LC/MS Procedure A) δ 7.61-7.58 (m, 1H), 7.48 (br s, 1H), 6.95 (s, 1H), 6.76 (d, J = 7.7 Hz, 1H), 6.54 (d, J = 7.7 Hz, 1H), 6.03 (brs, 2H), 5.98 (s, 1H), 5.61 (s, 2H), 5.24 (br s, 1H), 4.65 (br s, 2H), 4.44 (br s, 2H), 3.72 (s, 3H), 2.32 (s, 3H) |
| | {4-[(5-amino-7-{[(5-methyl-1,2- | | | |
| | oxazol-3-yl)methyl]amino}-1H- | | | |
| | pyrazolo[4,3-d]pyrimidin-1- | | | |
| | yl)methyl]-3-methoxyphenyl} | | | |
| | methanol | | | |
| 102 | 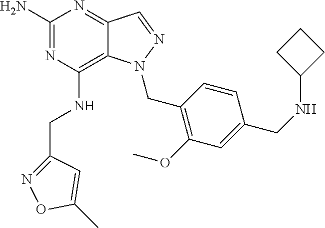 [SMILES: COc1cc(CNC2CCC2)ccc1Cn1ncc2nc(N)nc(NCc3cc(C)on3)c21] | 74.6 | 25.8 | LC/MS [M + H]⁺ = 449.2 RT (min) = 0.79 (LC/MS Procedure A) δ 9.35-9.14 (m, 2H), 8.78-8.70 (m, 1H), 7.75 (s, 1H), 7.73-7.65 (m, 1H), 7.18 (br s, 1H), 6.97 (br d, J = 8.0 Hz, 1H), 6.93-6.88 (m, 1H), 6.25 (s, 1H), 5.75-5.69 (m, 2H), 4.78 (br d,J = 3.3 Hz, 2H), 3.99 (br s, 2H), 3.71 (s, 3H), 3.67-3.61 (m, 1H), 2.40-2.33 (m, 3H), 2.20-2.10 (m, 4H), 1.82-1.72 (m, 2H) |
| | 1-({4-[(cyclobutylamino)methyl]- | | | |
| | 2-methoxyphenyl}methyl)-N7- | | | |
| | [(5-methyl-1,2-oxazol-3- | | | |
| | yl)methyl]-1H-pyrazolo[4,3- | | | |
| | d]pyrimidine-5,7-diamine | | | |
| 103 | 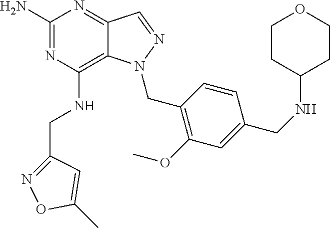 [SMILES: COc1cc(CNC2CCOCC2)ccc1Cn1ncc2nc(N)nc(NCc3cc(C)on3)c21] | 57.6 | 5.4 | LC/MS [M + H]⁺ = 479.2 RT (min) = 0.95 (LC/MS Procedure A) δ 7.72 (s, 1H), 7.20 (s, 1H), 7.00 (br d, J = 7.6 Hz, 1H), 6.87 (br d, J = 7.6 Hz, 1H), 6.23 (s, 1H), 5.71 (s, 2H), 4.76 (br s, 2H), 4.15 (s, 2H), 3.93 (br dd, J = 11.0, 3.4 Hz, 2H), 3.74 (s, 3H), 3.36-3.22 (m, 2H), 2.37 (s, 3H), 2.00 (br d, J = 11.6 Hz, 2H), 1.60 (qd, J = 12.2, 3.7 Hz, 2H) |
| | 1-[(2-methoxy-4-{[(oxan-4- | | | |
| | yl)amino]methyl}phenyl)methyl]- | | | |
| | N7-[(5-methyl-1,2-oxazol-3- | | | |
| | yl)methyl]-1H-pyrazolo[4,3- | | | |
| | d]pyrimidine-5,7-diamine | | | |
| 104 | 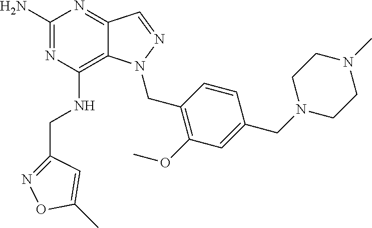 [SMILES: COc1cc(CN2CCN(C)CC2)ccc1Cn1ncc2nc(N)nc(NCc3cc(C)on3)c21] | 12.3 | 3.6 | LC/MS [M + H]⁺ = 478.2 RT (min) = 0.69 (LC/MS Procedure A) δ 8.89 (br t, J = 5.6 Hz, 1H), 8.03-7.93 (m, 1H), 7.78 (s, 1H), 6.96 (s, 1H), 6.91- 6.87 (m, 1H), 6.87-6.83 (m, 1H), 6.23 (s, 1H), 5.69 (s, 2H), 4.81 (br d, J = 5.8 Hz, 2H), 3.68 (s, 3H), 2.75 (s, 3H), 2.39- 2.35 (m, 3H) |
| | 1-({2-methoxy-4-[(4-methyl- | | | |
| | piperazin-1-yl)methyl]phenyl}- | | | |
| | methyl)-N7-[(5-methyl-1,2-oxazol- | | | |
| | 3-yl)methyl]-1H-pyrazolo[4,3- | | | |
| | d]pyrimidine-5,7-diamine | | | |
| 105 | 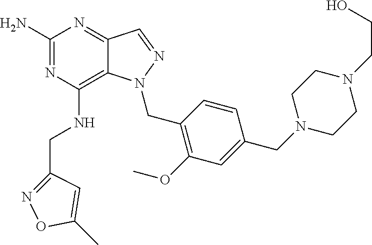 [SMILES: COc1cc(CN2CCN(CCO)CC2)ccc1Cn1ncc2nc(N)nc(NCc3cc(C)on3)c21] | 23.4 | | LC/MS [M + H]⁺ = 508.0 RT (min) = 1.00 (LC/MS Procedure A) δ 8.92-8.87 (m, 1H), 8.04-7.92 (m, 2H), 7.77 (s, 1H), 7.00 (s, 1H), 6.93- 6.86 (m, 2H), 6.22 (s, 1H), 5.69 (s, 2H), 4.79 (br d, J = 5.8 Hz, 2H), 3.75 (br s, 2H), 3.35-3.20 (m, 4H), 3.15-3.09 (m, 2H), 2.98-2.72 (m, 4H), 2.35 (s, 2H) |
| | 2-[4-({4-[(5-amino-7-{[(5-methyl- | | | |
| | 1,2-oxazol-3-yl)methyl]amino}- | | | |
| | 1H-pyrazolo[4,3-d]pyrimidin-1- | | | |
| | yl)methyl]-3-methoxyphenyl}- | | | |
| | methyl)piperazin-1-yl]ethan-1-ol | | | |
| 106 | 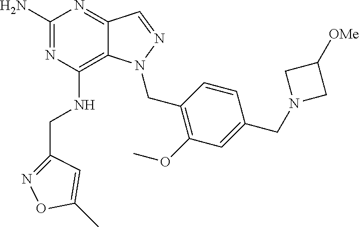 [SMILES: COc1cc(CN2CC(OC)C2)ccc1Cn1ncc2nc(N)nc(NCc3cc(C)on3)c21] | 57.0 | 9.5 | LC/MS [M + H]⁺ = 465.1 RT (min) = 1.00 (LC/MS Procedure A) δ 8.91-8.80 (m, 1H), 7.99-7.84 (m, 2H), 7.77 (s, 1H), 7.12 (s, 1H), 6.96 (br d, J = 6.9 Hz, 1H), 6.90 (d, J = 7.7 Hz, 1H), 6.23 (s, 1H), 5.73-5.69 (m, 2H), 4.78 (br d, J = 5.2 Hz, 2H), 4.32 (s, 2H), 4.22 (br d, J = 6.1 Hz, 3H), 3.92 (br d, J = 6.3 Hz, 2H), 3.71 (s, 3H), 3.23 (s, 3H), 2.36 (s, 3H) |
| | 1-({2-methoxy-4-[(3-methoxy- | | | |
| | azetidin-1-yl)methyl]phenyl}- | | | |
| | methyl)-N7-[(5-methyl-1,2-oxa- | | | |
| | zol-3-yl)methyl]-1H-pyrazolo- | | | |
| | [4,3-d]pyrimidine-5,7-diamine | | | |
| 107 | 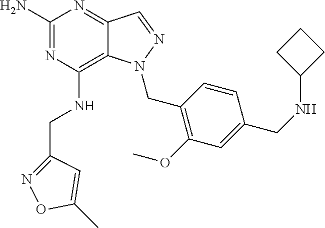 [SMILES: COc1cc(CNC2CCC2)ccc1Cn1ncc2nc(N)nc(NCc3cc(C)on3)c21] | 46.6 | 5.9 | LC/MS [M + H]⁺ = 450.3 RT (min) = 1.04 (LC/MS Procedure A) δ 7.59-7.55 (m, 1H), 7.37-7.29 (m, 1H), 7.00-6.98 (m, 1H), 6.78 (d, J = 7.4 Hz, 1H), 6.66 (d, J = 7.7 Hz, 1H), 5.66 (s, 2H), 5.59 (s, 2H), 4.77 (br s, 2H), 3.78 (s, 3H), 3.62 (s, 3H), 3.26-3.18 (m, 1H), 2.08-2.02 (m, 2H), 1.79-1.71 (m, 2H), 1.65-1.51 (m, 2H) |
| | 1-({4-[(cyclobutylamino)methyl]- | | | |
| | 2-methoxyphenyl}methyl)-N7- | | | |
| | [(5-methyl-1,2,4-oxadiazol-3- | | | |
| | yl)methyl]-1H-pyrazolo[4,3- | | | |
| | d]pyrimidine-5,7-diamine | | | |
| 108 | 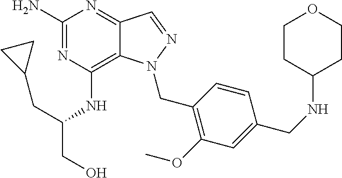 [SMILES: COc1cc(CNC2CCOCC2)ccc1Cn1ncc2nc(N)nc(N[C@H](CO)CC3CC3)c21] | 341.9 | 119.1 | LC/MS [M + H]⁺ = 482.3 RT (min) = 0.97 (LC/MS Method C) δ 7.58 (s, 1H), 7.09 (s, 1H), 6.82 (d, J = 7.8 Hz, 1H), 6.52 (d, J = 7.7 Hz, 1H), 5.82-5.71 (m, 1H), 5.63 (s, 2H), 5.50 (d, J = 16.8 Hz, 1H), 4.27 (s, 1H), 3.86 (s, 3H), 3.82 (d, J = 11.8 Hz, 2H), 3.74 (s, 1H), 3.24 (dd, J = 12.3, 10.0 Hz, 1H), 2.66 (m, 1H), 2.56 (s, 3H), 1.78 (d, J = 12.8 Hz, 2H), 1.34 (m, 4H), 0.45 (m, 1H), 0.26 (d, J = 8.1 Hz, 2H), −0.06 (s, 2H). |
| | (2S)-2-({5-amino-1-[(2-methoxy- | | | |
| | 4-{[(oxan-4- | | | |
| | yl)amino]methyl}phenyl)methyl]- | | | |
| | 1H-pyrazolo[4,3-d]pyrimidin-7- | | | |
| | yl}amino)-3-cyclopropylpropan- | | | |
| | 1-ol | | | |
| 109A | 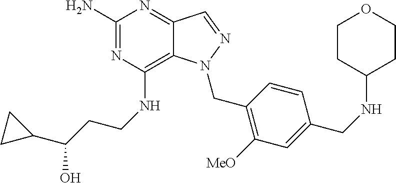 [SMILES: COc1cc(CNC2CCOCC2)ccc1Cn1ncc2nc(N)nc(NCC[C@H](O)C3CC3)c21] | 337.7 | 75.4 | LC/MS [M + H]⁺ = 482.3 RT (min) = 1.09 (LC/MS Procedure A) δ 7.50 (s, 1H), 7.00 (s, 1H), 6.79-6.71 (m, 1H), 6.46-6.38 (m, 1H), 5.64-5.57 (m, 1H), 5.55-5.48 (m, 2H), 3.80 (s, 4H), 3.67-3.60 (m, 1H), 3.27-3.15 (m, 2H), 2.85-2.75 (m, 1H), 1.82 (s, 8H), 1.75-1.67 (m, 3H), 1.63-1.54 (m, 1H), 1.28-1.15 (m, 3H), 0.79-0.68 (m, 1H), 0.35-0.24 (m, 2H), 0.19-0.10 (m, 1H), 0.05--0.05 (m, 1H) |
| | (1S)-3-({5-amino-1-[(2-methoxy- | | | |
| | 4-{[(oxan-4-yl)amino]methyl}- | | | |
| | phenyl)methyl]-1H-pyrazolo[4,3- | | | |
| | d]pyrimidin-7-yl}amino)-1- | | | |
| | cyclopropylpropan-1-ol | | | |
| 109b | 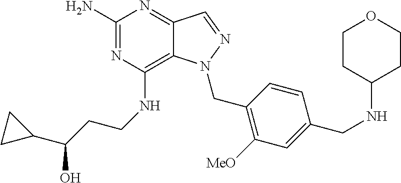 [SMILES: COc1cc(CNC2CCOCC2)ccc1Cn1ncc2nc(N)nc(NCC[C@@H](O)C3CC3)c21] | 1,333 | 162.7 | LC/MS [M + H]⁺ = 482.1 RT (min) = 1.09 (LC/MS Procedure A) δ 7.50 (s, 1H), 7.00 (s, 1H), 6.80-6.70 (m, 1H), 6.45-6.36 (m, 1H), 5.59 (s, 1H), 5.52 (s, 2H), 3.79 (s, 4H), 3.68- 3.60 (m, 1H), 3.19 (s, 2H), 2.84-2.75 (m, 1H), 2.48-2.43 (m, 5H), 1.86 (s, 3H), 1.77-1.66 (m, 3H), 1.63-1.53 (m, 1H), 1.30-1.15 (m, 3H), 0.79-0.68 (m, 1H), 0.33-0.23 (m, 2H), 0.19-0.09 (m, 1H), 0.04--0.04 (m, 1H) |
| | (1R)-3-({5-amino-1-[(2-methoxy-4- | | | |
| | {[(oxan-4-yl)amino]methyl}- | | | |
| | phenyl)methyl]-1H-pyrazolo[4,3- | | | |
| | d]pyrimidin-7-yl}amino)-1- | | | |
| | cyclopropylpropan-1-ol | | | |
| 110 | 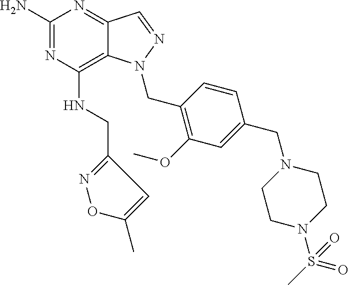 [SMILES: COc1cc(CN2CCN(S(C)(=O)=O)CC2)ccc1Cn1ncc2nc(N)nc(NCc3cc(C)on3)c21] | 485.3 | 438.2 | LC/MS [M + H]⁺ = 542.2 RT (min) = 1.01 (LC/MS Procedure A) δ 8.86 (br t, J = 5.1 Hz, 1H), 7.96-7.88 (m, 1H), 7.79-7.76 (m, 1H), 7.27 (s, 1H), 7.17 (s, 1H), 7.06 (s, 1H), 7.05- 7.03 (m, 1H), 6.90 (s, 2H), 6.22 (s, 1H), 5.70 (s, 2H), 4.79 (br d, J = 5.5 Hz, 2H), 3.69 (s, 3H), 3.23 (br d, J = 6.9 Hz, 4H), 2.90 (s, 3H), 2.89-2.79 (m, 4H), 2.36 (s, 3H). |
| | 1-({4-[(4-methanesulfonyl- | | | |
| | piperazin-1-yl)methyl]-2- | | | |
| | methoxyphenyl}methyl)-N7-[(5- | | | |
| | methyl-1,2-oxazol-3-yl)methyl]- | | | |
| | 1H-pyrazolo[4,3-d]pyrimidine- | | | |
| | 5,7-diamine | | | |
| 111 | 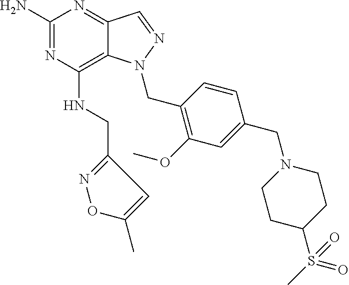 [SMILES: COc1cc(CN2CCC(S(C)(=O)=O)CC2)ccc1Cn1ncc2nc(N)nc(NCc3cc(C)on3)c21] | 176 | 58.8 | LC/MS [M + H]⁺ = 541.2 RT (min) = 0.99 (LC/MS Procedure A) δ 8.82 (br s, 1H), 7.94-7.82 (m, 2H), 7.79-7.76 (m, 1H), 7.12 (s, 1H), 6.98- 6.94 (m, 1H), 6.93-6.87 (m, 1H), 6.22 (s, 1H), 5.72 (s, 2H), 4.78 (br d, J = 4.4 Hz, 2H), 4.21-4.11 (m, 2H), 3.71 (s, 3H), 3.40-3.33 (m, 4H), 2.96 (s, 3H), 2.88-2.77 (m, 1H), 2.35 (s, 3H), 2.18 (brd, J = 13.2 Hz, 2H), 1.89-1.74 (m, 2H) |
| | 1-({4-[(4-methanesulfonylpiper- | | | |
| | idin-1-yl)methyl]-2-methoxy- | | | |
| | phenyl}ol-3-yl)methyl]-1H-pyra- | | | |
| | zolo[4,3-d]pyrimidine-5,7- | | | |
| | diamine | | | |
| 112 | 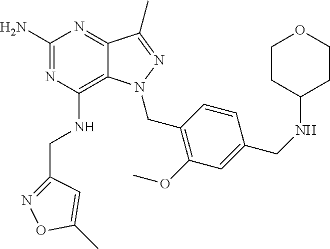 [SMILES: COc1cc(CNC2CCOCC2)ccc1Cn1nc(C)c2nc(N)nc(NCc3cc(C)on3)c21] | 95.9 | 10.3 | [M + H]⁺ = 493.2 LC RT = 1.03 (LC/MS Method C) δ 7.24 (br t, J = 5.8 Hz, 1H), 7.02 (s, 1H), 6.80 (br d, J = 7.6 Hz, 1H), 6.49 (br d, J = 7.6 Hz, 1H), 6.02 (s, 1H), 5.75 (s, 2H), 5.53 (s, 2H), 4.65 (br d, J = 5.2 Hz, 2H), 3.82 (br d, J = 11.0 Hz, 2H), 3.78-3.70 (m, 3H), 3.25 (br t, J = 10.8 Hz, 2H), 2.66- 2.59 (m, 1H), 2.35 (s, 3H), 2.25 (s, 3H), 1.77 (br d, J = 12.8 Hz, 2H), 1.36-1.22 (m, 2H) |
| | 1-[(2-methoxy-4-{[(oxan-4- | | | |
| | yl)amino]methyl}phenyl)methyl]- | | | |
| | 3-methyl-N7-[(5-methyl-1,2-oxa- | | | |
| | zol-3-yl)methyl]-1H-pyrazolo- | | | |
| | [4,3-d]pyrimidine-5,7-diamine | | | |
| 113 | 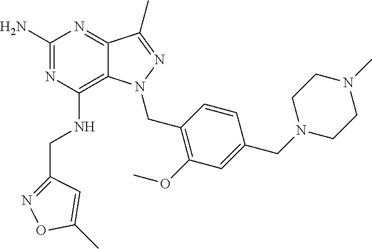 [SMILES: COc1cc(CN2CCN(C)CC2)ccc1Cn1nc(C)c2nc(N)nc(NCc3cc(C)on3)c21] | 100.4 | 40.5 | [M + H]⁺ = 492.2 LC RT = 1.01 (LC/MS Method C) δ 6.91 (s, 1H), 6.73 (d, J = 7.7 Hz, 1H), 6.45 (d, J = 7.6 Hz, 1H), 5.99 (s, 1H), 5.75 (br s, 2H), 5.52 (s, 2H), 4.63 (br d, J = 5.4 Hz, 2H), 3.75 (s, 3H), 3.17 (s, 2H), 2.40- 2.27 (m, 7H), 2.23 (s, 4H), 2.14 (s, 3H), 1.91 (s, 3H) |
| | 1-({2-methoxy-4-[(4-methylpipe- | | | |
| | azin-1-yl)methyl]phenyl}methyl)- | | | |
| | 3-methyl-N7-[(5-methyl-1,2- | | | |
| | oxazol-3-yl)methyl]-1H- | | | |
| | pyrazolo[4,3-d]pyrimidine-5,7- | | | |
| | diamine | | | |
| 114 | 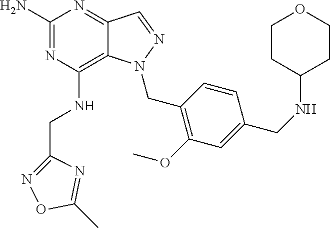 [SMILES: COc1cc(CNC2CCOCC2)ccc1Cn1ncc2nc(N)nc(NCc3noc(C)n3)c21] | 79.1 | 19.6 | LC/MS [M + H]⁺ = 479.9 RT (min) = 0.98 (LC/MS Procedure A) δ 7.59-7.55 (m, 1H), 7.36-7.30 (m, 1H), 7.01 (s, 1H), 6.80 (brd, J = 7.4 Hz, 1H), 6.65 (d, J = 7.4 Hz, 1H), 5.66 (s, 2H), 5.58 (s, 2H), 4.77 (br s, 2H), 3.81 (br s, 2H), 3.78 (s, 3H), 3.70 (s, 2H), 3.23 (br t, J = 10.7 Hz, 2H), 2.61-2.57 (m, 1H), 2.55 (s, 3H), 1.75 (br d, J = 12.7 Hz, 2H), 1.33-1.22 (m, 2H) |
| | 1-[(2-methoxy-4-{[(oxan-4- | | | |
| | yl)amino]methyl}phenyl)methyl]- | | | |
| | N7-[(5-methyl-1,2,4-oxadiazol- | | | |
| | 3-yl)methyl]-1H-pyrazolo[4,3- | | | |
| | d]pyrimidine-5,7-diamine | | | |
| 115 | 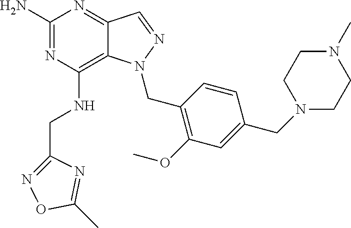 [SMILES: COc1cc(CN2CCN(C)CC2)ccc1Cn1ncc2nc(N)nc(NCc3noc(C)n3)c21] | 67.2 | 15.5 | LC/MS [M + H]⁺ = 479.2 RT (min) = 0.96 (LC/MS Procedure A) δ 7.56 (s, 1H), 6.92-6.90 (m, 1H), 6.74 (br d, J = 7.6 Hz, 1H), 6.65 (d, J = 7.6 Hz, 1H), 5.64 (s, 2H), 5.57 (s, 2H), 4.77 (br s, 2H), 2.53 (br s, 3H), 2.39-2.22 (m, 6H), 2.11 (s, 3H) |
| | 1-({2-methoxy-4-[(4-methylpiper- | | | |
| | azin-1-yl)methyl]phenyl} | | | |
| | methyl)-N7-[(5-methyl-1,2,4-oxadi- | | | |
| | azol-3-yl)methyl]-1H-pyrazolo- | | | |
| | [4,3-d]pyrimidine-5,7-diamine | | | |
| 116 | 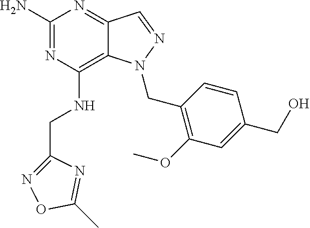 [SMILES: COc1cc(CO)ccc1Cn1ncc2nc(N)nc(NCc3noc(C)n3)c21] | 728.9 | | LC/MS [M + H]⁺ = 397.1 RT (min) = 1.01 (LC/MS Procedure A) δ 7.58-7.54 (m, 1H), 7.43-7.25 (m, 1H), 6.94 (s, 1H), 6.76 (d, J = 7.7 Hz, 1H), 6.68 (br d, J = 7.9 Hz, 1H), 5.65 (s, 2H), 5.58 (s, 2H), 4.77 (br s, 2H), 4.44 (s, 2H), 3.77 (s, 3H), 3.16 (s, 1H) |
| | {4-[(5-amino-7-{[(5-methyl-1,2,4- | | | |
| | oxadiazol-3-yl)methyl]amino}- | | | |
| | 1H-pyrazolo[4,3-d]pyrimidin-1- | | | |
| | yl)methyl]-3- | | | |
| | methoxyphenyl}methanol | | | |
| 117 | 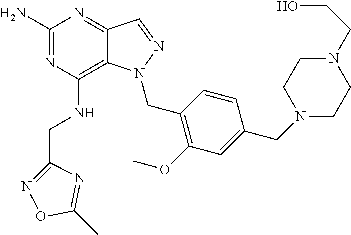 [SMILES: COc1cc(CN2CCN(CCO)CC2)ccc1Cn1ncc2nc(N)nc(NCc3noc(C)n3)c21] | 18.6 | 1.4 | LC/MS [M + H]⁺ = 509.3 RT (min) = 0.52/Method D ¹H NMR (400 MHz, METHANOL-d₄) δ 7.73-7.68 (m, 1H), 7.28 (br s, 1H), 7.21-7.14 (m, 1H), 7.08 (br d, J = 7.5 Hz, 1H), 5.76 (s, 2H), 4.99 (s, 2H), 4.34- 4.08 (m, 2H), 3.91-3.87 (m, 5H), 3.35- 3.32 (m, 5H), 2.69 (s, 4H), 2.59 (s, 3H). |
| | 2-[4-({4-[(5-amino-7-{[(5-methyl- | | | |
| | 1,2,4-oxadiazol-3-yl)methyl]ami- | | | |
| | no}-1H-pyrazolo[4,3-d]pyrimidin-1- | | | |
| | yl)methyl]-3-methoxyphenyl}- | | | |
| | methyl)piperazin-1-yl]ethan-1-ol | | | |
| 118 | 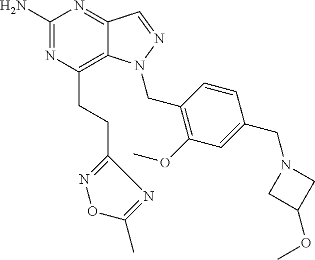 [SMILES: COc1cc(CN2CC(OC)C2)ccc1Cn1ncc2nc(N)nc(CCc3noc(C)n3)c21] | 47.9 | 4.3 | LC/MS [M + H]⁺ = 466.2 RT (min) = 0.99/Method C δ 7.83-7.73 (m, 1H), 7.13 (s, 1H), 7.03- 6.93 (m, 2H), 5.72 (s, 2H), 4.91 (br s, 2H), 4.32 (s, 2H), 4.27-4.19 (m, 3H), 3.92 (br d, J = 6.7 Hz, 2H), 3.76 (s, 3H), 3.23 (s, 3H), 2.58 (s, 3H) |
| | 1-({2-methoxy-4-[(3- | | | |
| | methoxyazetidin-1- | | | |
| | yl)methyl]phenyl}methyl)-N7-[(5- | | | |
| | methyl-1,2,4-oxadiazol-3- | | | |
| | yl)methyl]-1H-pyrazolo[4,3- | | | |
| | d]pyrimidine-5,7-diamine | | | |
| 119 | 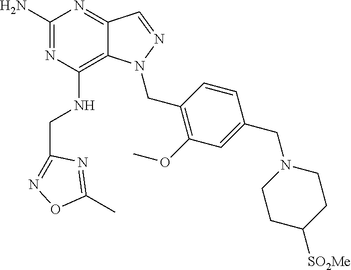 [SMILES: COc1cc(CN2CCC(S(C)(=O)=O)CC2)ccc1Cn1ncc2nc(N)nc(NCc3noc(C)n3)c21] | 298.5 | 34.7 | LC/MS [M + H]⁺ = 541.9 RT (min) = 0.99/Method C δ 9.02-8.95 (m, 1H), 8.06-7.88 (m, 2H), 7.80 (s, 1H), 7.17 (s, 1H), 7.02- 6.99 (m, 2H), 5.78-5.71 (m, 2H), 4.92 (br d, J = 5.5 Hz, 2H), 4.29-4.26 (m, 1H), 4.26 (br s, 2H), 3.77 (s, 3H), 2.98 (s, 3H), 2.94 (br s, 1H), 2.57 (s, 3H), 2.22 (brd, J = 12.7 Hz, 2H), 1.94-1.79 (m, 2H) |
| | 1-({4-[(4-methanesulfonyl- | | | |
| | piperidin-1-yl)methyl]-2- | | | |
| | methoxyphenyl}methyl)-N7-[(5- | | | |
| | methyl-1,2,4-oxadiazol-3- | | | |
| | yl)methyl]-1H-pyrazolo[4,3- | | | |
| | d]pyrimidine-5,7-diamine | | | |
| 120 | 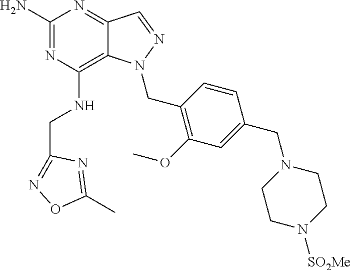 [SMILES: COc1cc(CN2CCN(S(C)(=O)=O)CC2)ccc1Cn1ncc2nc(N)nc(NCc3noc(C)n3)c21] | 344.5 | 298.55 | LC/MS [M + H]⁺ = 543.2 RT (min) = 1.05/Method C δ 9.02-8.95 (m, 1H), 8.06-7.88 (m, 2H), 7.80 (s, 1H), 7.17 (s, 1H), 7.02- 6.99 (m, 2H), 5.78-5.71 (m, 2H), 4.92 (br d, J = 5.5 Hz, 2H), 4.29-4.26 (m, 1H), 4.26 (br s, 2H), 3.77 (s, 3H), 2.98 (s, 3H), 2.94 (br s, 1H), 2.57 (s, 3H), 2.22 (brd, J = 12.7 Hz, 2H), 1.94-1.79 (m, 2H) |
| | 1-({4-[(4-methanesulfonyl- | | | |
| | piperazin-1-yl)methyl]-2- | | | |
| | methoxyphenyl}methyl)-N7-[(5- | | | |
| | methyl-1,2,4-oxadiazol-3- | | | |
| | yl)methyl]-1H-pyrazolo[4,3- | | | |
| | d]pyrimidine-5,7-diamine | | | |
| 121 | 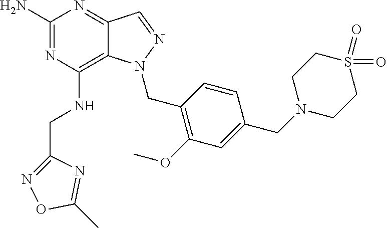 [SMILES: COc1cc(CN2CCS(=O)(=O)CC2)ccc1Cn1ncc2nc(N)nc(NCc3noc(C)n3)c21] | 655.7 | 288.6 | LC/MS [M + H]⁺ = 514.1 RT (min) = 1.0/Method C δ 7.58 (s, 1H), 7.39 (br d, J = 6.3 Hz, 1H), 6.97 (s, 1H), 6.79 (br d, J = 7.7 Hz, 1H), 6.66 (d, J = 7.7 Hz, 1H), 5.73 (br s, 2H), 5.60 (s, 2H), 4.78 (br d, J = 5.2 Hz, 2H), 3.79 (s, 3H), 3.61 (s, 2H), 3.08 (br s, 4H), 2.84 (br s, 4H), 2.56-2.54 (m, 3H) |
| | 4-({4-[(5-amino-7-{[(5-methyl- | | | |
| | 1,2,4-oxadiazol-3-yl)methyl]- | | | |
| | amino}-1H-pyrazolo[4,3- | | | |
| | d]pyrimidin-1-yl)methyl]-3- | | | |
| | methoxyphenyl}methyl)-1λ⁶- | | | |
| | thiomorpholine-1,1-dione | | | |
| 122 | 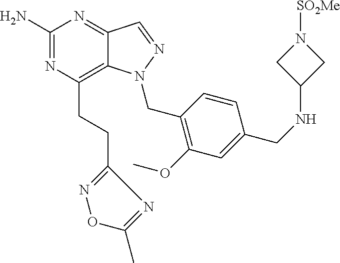 [SMILES: COc1cc(CNC2CN(S(C)(=O)=O)C2)ccc1Cn1ncc2nc(N)nc(CCc3noc(C)n3)c21] | 570.4 | 220.9 | LC/MS [M + H]⁺ = 529.1 RT (min) = 0.96/Method C δ 7.57 (s, 1H), 7.33 (br t, J = 5.8 Hz, 1H), 6.99-6.96 (m, 1H), 6.77 (d, J = 7.8 Hz, 1H), 6.64 (d, J = 7.6 Hz, 1H), 5.68 (s, 2H), 5.59 (s, 2H), 4.77 (br d, J = 5.2 Hz, 2H), 3.86-3.81 (m, 2H), 3.79 (s, 3H), 3.59 (s, 2H), 3.58-3.54 (m, 2H), 3.50 (dt, J = 13.1, 6.8 Hz, 2H), 2.94 (s, 3H), 2.56 (s, 3H) |
| | 1-[(4-{[(1-methanesulfonylazetidin- | | | |
| | 3-yl)amino]methyl}-2-methoxy- | | | |
| | phenyl)methyl]-N7-[(5-methyl- | | | |
| | 1,2,4-oxadiazol-3-yl)methyl]-1H- | | | |
| | pyrazolo[4,3-d]pyrimidine-5,7- | | | |
| | diamine | | | |
| 123 | 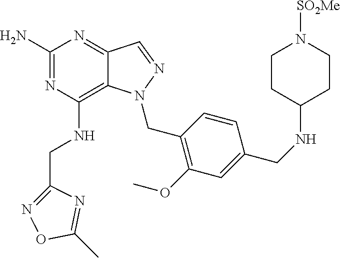 [SMILES: COc1cc(CNC2CCN(S(C)(=O)=O)CC2)ccc1Cn1ncc2nc(N)nc(NCc3noc(C)n3)c21] | 195.8 | 18.2 | LC/MS [M + H]⁺ = 557.2 RT (min) = 0.97/Method C δ 7.81-7.78 (m, 1H), 7.23 (s, 1H), 7.09- 7.03 (m, 2H), 5.76 (s, 2H), 4.95 (br d, J = 5.2 Hz, 2H), 4.20 (br s, 2H), 3.78 (s, 3H), 3.66 (br d, J = 12.2 Hz, 2H), 3.26- 3.18 (m, 1H), 2.90 (s, 3H), 2.79 (brt, J = 11.6 Hz, 2H), 2.61 (s, 3H), 2.20 (br d, J = 11.6 Hz, 2H), 1.70-1.61 (m, 2H) |
| | 1-[(4-{[(1-methanesulfonyl- | | | |
| | piperidin-4-yl)amino]methyl}-2- | | | |
| | methoxyphenyl)methyl]-N7-[(5- | | | |
| | methyl-1,2,4-oxadiazol-3- | | | |
| | yl)methyl]-1H-pyrazolo[4,3- | | | |
| | d]pyrimidine-5,7-diamine | | | |
| 125 | 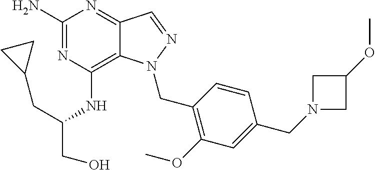 [SMILES: COc1cc(CN2CC(OC)C2)ccc1Cn1ncc2nc(N)nc(N[C@H](CO)CC3CC3)c21] (2S)-2-{[5-amino-1-({2-methoxy-4- | 137.8 | 15.2 | LC/MS [M + H]⁺ = 468.2 RT (min) = 1.16 (Method B) δ 7.89 (br d, J = 7.6 Hz, 1H), 7.81 (s, 1H), 7.45 (br d, J = 7.9 Hz, 1H), 7.20 (s, 1H), 6.99 (br d, J = 8.5 Hz, 1H), 6.83 (br d, J = 7.9 Hz, 1H), 5.90-5.65 (m, 2H), 4.60- 4.44 (m, 1H), 4.40-4.31 (m, 2H), 4.28- 4.14 (m, 3H), 4.01-3.87 (m, 2H), 3.83 (s, 2H), 3.68-3.50 (m, 1H), 3.30-3.22 (m, 2H), 1.65-1.38 (m, 3H), 0.63-0.49 (m, 1H), 0.39-0.22 (m, 2H), 0.01 (br d, J = 4.3 Hz, 2H) |
| | [(3-methoxyazetidin-1-yl)methyl] | | | |
| | phenyl}methyl)-1H-pyrazolo- | | | |
| | [4,3-d]pyrimidin-7-yl]amino}-3- | | | |
| | cyclopropylpropan-1-ol | | | |
| 126 | 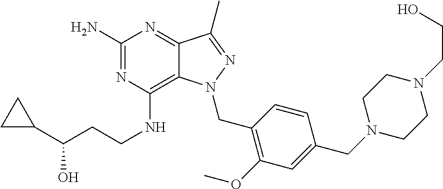 [SMILES: COc1cc(CN2CCN(CCO)CC2)ccc1Cn1nc(C)c2nc(N)nc(NCC[C@H](O)C3CC3)c21] (1S)-3-({5-amino-1-[(4-{[4-(2- | 739.7 | 25.3 | LC/MS [M + H]⁺ = 525.3 RT (min) = 1.14 (LC/MS Procedure A) δ 6.82-6.73 (m, 1H), 6.61-6.50 (m, 1H), 6.41-6.34 (m, 1H), 6.30-6.20 (m, 1H), 5.61-5.46 (m, 2H), 5.35-5.23 (m, 2H), 3.65 (s, 3H), 3.42-3.19 (m, 1H), 2.75-2.59 (m, 1H), 2.35-2.31 (m, 6H), 2.30-2.26 (m, 1H), 2.25-2.20 (m, 2H), 2.20-2.13 (m, 2H), 2.05 (s, 4H), 1.73 (s, 3H), 1.64-1.52 (m, 1H), 1.49-1.36 (m, 1H), 0.69-0.51 (m, 1H), 0.20-0.07 (m, 2H), 0.05--0.06 (m, 1H), −0.09-- 0.23 (m, 1H) |
| | hydroxyethyl)piperazin-1- | | | |
| | yl]methyl}-2-methoxyphenyl)- | | | |
| | methyl]-3-methyl-1H-pyrazolo[4,3- | | | |
| | d]pyrimidin-7-yl}amino)-1- | | | |
| | cyclopropylpropan-1-ol | | | |
| 127 | 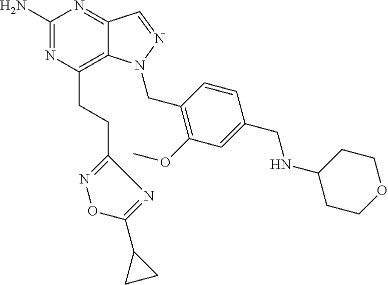 [SMILES: COc1cc(CNC2CCOCC2)ccc1Cn1ncc2nc(N)nc(CCc3noc(C4CC4)n3)c21] | 72.4 | 25.5 | LC/MS [M + H]⁺ = 505.9 RT (min) = 1.03/Method C δ 7.57 (s, 1H), 7.29 (br s, 1H), 7.01 (br s, 1H), 6.80 (br d, J = 7.4 Hz, 1H), 6.64 (br d, J = 7.4 Hz, 1H), 5.67 (br s, 2H), 5.59 (br s, 2H), 4.74 (br d, J = 3.0 Hz, 2H), 3.81 (br s, 2H), 3.79 (s, 3H), 3.71 (br s, 2H), 3.23 (br t, J = 11.4 Hz, 2H), 2.65-2.56 (m, 1H), 2.33-2.25 (m, 1H), 1.76 (br d, J = 13.5 Hz, 2H), 1.32-1.25 (m, 2H), 1.22 (br dd, J = 8.1, 2.3 Hz, 2H), 1.12-1.00 (m, 2H) |
| | N7-[(5-cyclopropyl-1,2,4-oxadiazol- | | | |
| | 3-yl)methyl]-1-[(2-methoxy-4- | | | |
| | {[(oxan-4- | | | |
| | yl)amino]methyl}phenyl)methyl]- | | | |
| | 1H-pyrazolo[4,3-d]pyrimidine-5,7- | | | |
| | diamine | | | |
| 128 | 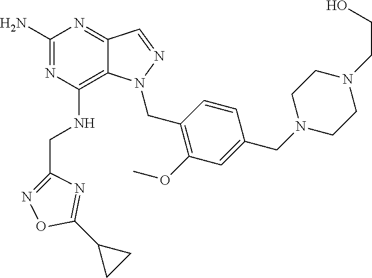 [SMILES: COc1cc(CN2CCN(CCO)CC2)ccc1Cn1ncc2nc(N)nc(NCc3noc(C4CC4)n3)c21] | 33.7 | 7.5 | LC/MS [M + H]⁺ = 535.2 RT (min) = 1.03/method C δ 7.57 (s, 1H), 7.31-7.24 (m, 1H), 6.94- 6.89 (m, 1H), 6.74 (br d, J = 7.9 Hz, 1H), 6.61 (d, J = 7.7 Hz, 1H), 5.66 (s, 2H), 5.58 (s, 2H), 4.73 (br d, J = 3.1 Hz, 2H), 3.77 (s, 3H), 3.38 (s, 2H), 2.46-2.23 (m, 11H), 1.26-1.17 (m, 2H), 1.06-1.00 (m, 2H) |
| | 2-[4-({4-[(5-amino-7-{[(5-cyclo- | | | |
| | propyl-1,2,4-oxadiazol-3-yl) | | | |
| | methyl]amino}-1H-pyrazolo[4,3- | | | |
| | d]pyrimidin-1-yl)methyl]-3- | | | |
| | methoxyphenyl}methyl)piperazin- | | | |
| | 1-yl]ethan-1-ol | | | |
| 129 | 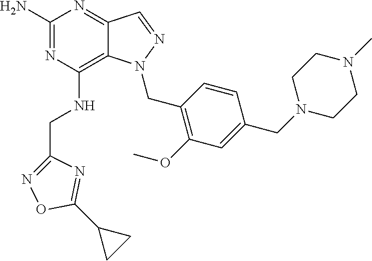 [SMILES: COc1cc(CN2CCN(C)CC2)ccc1Cn1ncc2nc(N)nc(NCc3noc(C4CC4)n3)c21] | 47.8 | 5.3 | LC/MS [M + H]⁺ = 505.1 RT (min) = 1.11/Method C δ 7.57 (s, 1H), 7.30 (br s, 1H), 6.94- 6.91 (m, 1H), 6.75 (br d, J = 7.9 Hz, 1H), 6.63 (d, J = 7.5 Hz, 1H), 5.68 (br s, 2H), 5.58 (s, 2H), 4.73 (br d, J = 4.0 Hz, 2H), 3.77 (s, 3H), 3.44 (br s, 2H), 3.41 (s, 2H), 2.48-2.46 (m, 1H), 2.45-2.35 (m, 4H), 2.28 (td, J = 8.6, 4.5 Hz, 2H), 2.24 (s, 3H), 1.25-1.18 (m, 2H), 1.03 (br dd, J = 4.5, 2.9 Hz, 2H) |
| | N7-[(5-cyclopropyl-1,2,4-oxadiazol- | | | |
| | 3-yl)methyl]-1-({2-methoxy-4-[(4- | | | |
| | methylpiperazin-1-yl)methyl]- | | | |
| | phenyl}methyl)-1H-pyrazolo[4,3- | | | |
| | d]pyrimidine-5,7-diamine | | | |
| 130 | 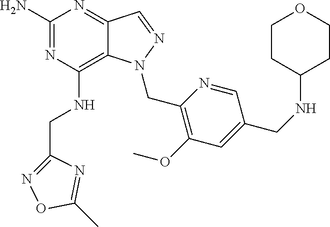 [SMILES: COc1cc(CNC2CCOCC2)cnc1Cn1ncc2nc(N)nc(NCc3noc(C)n3)c21] | 1,350 | 85 | LC/MS [M + H]⁺ = 480.9 RT (min) = 0.94/Method C δ 8.15-8.08 (m, 1H), 7.79 (s, 1H), 7.75 (s, 1H), 5.89 (s, 2H), 4.94 (br d, J = 5.5 Hz, 2H), 4.25 (s, 2H), 3.98-3.93 (m, 2H), 3.92 (s, 3H), 3.33 (brt, J = 11.7 Hz, 2H), 2.62 (s, 3H), 2.02 (br dd, J = 11.7, 1.4 Hz, 2H), 1.69-1.55 (m, 2H) |
| | 1-[(3-methoxy-5-{[(oxan-4-yl) | | | |
| | amino]methyl}pyridin-2-yl)methyl]- | | | |
| | N7-[(5-methyl-1,2,4-oxadiazol-3- | | | |
| | yl)methyl]-1H-pyrazolo[4,3- | | | |
| | d]pyrimidine-5,7-diamine | | | |
| 131 | 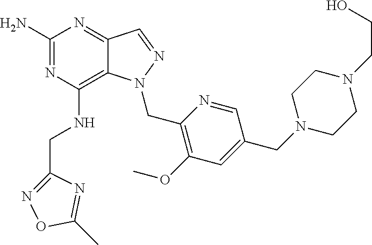 [SMILES: COc1cc(CN2CCN(CCO)CC2)cnc1Cn1ncc2nc(N)nc(NCc3noc(C)n3)c21] | 1,000 | 72.7 | LC/MS [M + H]⁺ = 510.3 RT (min) = 0.47/Method D ¹H NMR (400 MHz, DMSO-d₆) δ 8.12- 8.05 (m, 1H), 7.99-7.90 (m, 1H), 7.81 (s, 1H), 5.87 (s, 2H), 4.93 (d, J = 5.4 Hz, 2H), 3.93 (s, 3H), 3.80-3.74 (m, 2H), 3.69-3.54 (m, 2H), 3.48-3.04 (m, 6H), 2.60 (s, 3H), 2.54-2.51 (m, 2H) |
| | 2-[4-({6-[(5-amino-7-{[(5-methyl- | | | |
| | 1,2,4-oxadiazol-3-yl)methyl]- | | | |
| | amino}-1H-pyrazolo[4,3- | | | |
| | d]pyrimidin-1-yl)methyl]-5- | | | |
| | methoxypyridin-3-yl}methyl)- | | | |
| | piperazin-1-yl]ethan-1-ol | | | |
| 132 | 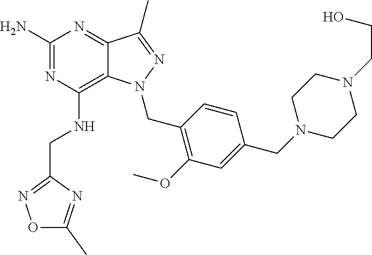 [SMILES: COc1cc(CN2CCN(CCO)CC2)ccc1Cn1nc(C)c2nc(N)nc(NCc3noc(C)n3)c21] | 423.82 | | LC/MS m/z = 523.2 [M + H]⁺ RT (min) = 0.849 (LC/MS method E) ¹H NMR (400 MHz, DMSO-d₆) δ = 7.26 (br s, 1H), 6.92 (s, 1H), 6.75 (d, J = 7.8 Hz, 1H), 6.62 (d, J = 7.8 Hz, 1H), 5.72 (s, 2H), 5.52 (s, 2H), 4.78 (br d, J = 2.0 Hz, 2H), 3.91 (s, 2H), 3.79 (s, 3H), 3.50- 3.47 (m, 2H), 3.40 (s, 2H), 2.56 (s, 3H), 2.48-2.30 (m, 8H), 2.23 (s, 3H). |
| | 2-[4-({4-[(5-amino-3-methyl-7-{[(5- | | | |
| | methyl-1,2,4-oxadiazol-3-yl) | | | |
| | methyl]amino}-1H-pyrazolo[4,3- | | | |
| | d]pyrimidin-1-yl)methyl]-3- | | | |
| | methoxyphenyl}methyl)piperazin- | | | |
| | 1-yl]ethan-1-ol | | | |
| 133 | 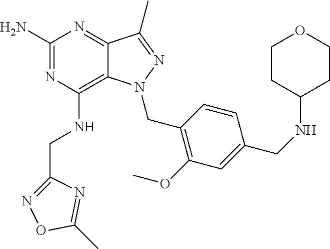 [SMILES: COc1cc(CNC2CCOCC2)ccc1Cn1nc(C)c2nc(N)nc(NCc3noc(C)n3)c21] | 1112 | | LC/MS m/z = 494.2 [M + H]⁺ RT (min) = 0.858 (LC/MS method E) ¹H NMR (400 MHz, DMSO-d₆) δ = 7.29 (br t, J = 5.7 Hz, 1H), 7.04 (s, 1H), 6.83 (br d, J = 1.0 Hz, 1H), 6.66 (d, J = 7.6 Hz, 1H), 5.69 (s, 2H), 5.53 (s, 2H), 4.78 (d, J = 5.6 Hz, 2H), 3.81 (m, 7H), 3.26-3.21 (m, 3H), 2.56 (s, 3H), 2.22 (s, 3H), 1.85- 1.77 (m, 2H), 1.41-1.28 (m, 2H). |
| | 1-[(2-methoxy-4-{[(oxan-4-yl) | | | |
| | amino]methyl}phenyl)methyl]-3- | | | |
| | methyl-N7-[(5-methyl-1,2,4-oxa- | | | |
| | diazol-3-yl)methyl]-1H-pyrazolo- | | | |
| | [4,3-d]pyrimidine-5,7-diamine | | | |
| 134 | 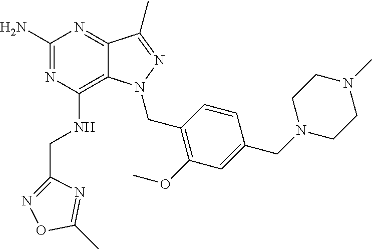 [SMILES: COc1cc(CN2CCN(C)CC2)ccc1Cn1nc(C)c2nc(N)nc(NCc3noc(C)n3)c21] | 159.4 | | LC/MS m/z = 493.2 [M + H]⁺ RT (min) = 0.876 (LC/MS method E) ¹H NMR (400 MHz, DMSO-d₆) δ = 7.25 (t, J = 5.3 Hz, 1H), 6.92 (d, J = 1.5 Hz, 1H), 6.79-6.70 (m, 1H), 6.61 (d, J = 7.6 Hz, 1H), 5.69 (s, 2H), 5.52 (s, 2H), 4.78 (d, J = 5.9 Hz, 2H), 3.79 (s, 3H), 3.40 (s, 2H), 2.56 (s, 3H), 2.38-2.29 (m, 8H), 2.23 (s, 3H), 2.16 (s, 3H). |
| | 1-({2-methoxy-4-[(4-methyl- | | | |
| | piperazin-1-yl)methyl]phenyl}- | | | |
| | methyl)-3-methyl-N7-[(5-methyl- | | | |
| | 1,2,4-oxadiazol-3-yl)methyl]-1H- | | | |
| | pyrazolo[4,3-d]pyrimidine-5,7- | | | |
| | diamine | | | |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### Industrial Applicability and Uses

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675, 206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and analytical (LC/MS) liquid chromatography methods were used:

LC/MS Method A: Column: BEH C18 2.1×50 mm; Mobile Phase A: water with 0.05% TFA; Mobile Phase B: acetonitrile with 0.05% TFA; Temperature: 50° C.; Gradient: 2-98% B over 1.7 min; Flow: 0.8 mL/min.

LC/MS Method B: Column: BEH C18 2.1×50 mm; Mobile Phase A: 95:5 H₂O:acetonitrile with 0.01M NH₄OAc; Mobile Phase B: 5:95 H₂O:acetonitrile with 0.01M NH₄OAc; Temperature: 50° C.; Gradient: 5-95% B over 1 min; Flow: 0.8 mL/min.

LC/MS Method C: Column: Waters XBridge C18, 2.1 mm×50 mm, 1.7 μm particles; Mobile Phase A: 5:95 acetonitrile:water with 0.1% TFA; Mobile Phase B: 95:5 acetonitrile:water with 0.1% TFA; Temperature: 50° C.; Gradient: 0% B to 100% B over 3 min, then a 0.50 min hold at 100% B; Flow: 1 mL/min; Detection: MS and UV (220 nm).

LC/MS Method D. Column: BEH C18 2.1×50 mm; Mobile Phase A: water with 0.05% TFA; Mobile Phase B: acetonitrile with 0.05% TFA; Temperature: 50° C.; Gradient: 2-98% B over 1.0 min, then a 0.50 min hold at 98% B; Flow: 0.8 mL/min. Detection: MS and UV (220 nm).

LCMS Method E. Column: Xbridge BEH C18 XP (50×2.1 mm), 2.5 μm; mobile phase A: 5:95 CH3CN: H2O with 10 mM NH4OAc; mobile phase B: 95:5 CH3CN: H2O with 10 mM NH4OAc; temperature: 50° C.; gradient: 0-100% B over 3 minutes; flow rate: 1.1 mL/min).

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

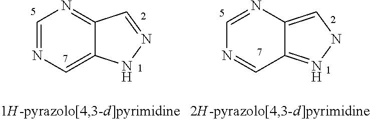 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety by reference.

The compounds of this invention may be prepared using the reactions and techniques described in this section. The reactions are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Third Edition, Wiley and Sons, 1999).

Compounds of Formula (I) may be prepared by reference to the methods illustrated in the following Schemes. As shown therein the end product is a compound having the same structural formula as Formula (I). It will be understood that any compound of Formula (I) may be produced by the schemes by the suitable selection of reagents with appropriate substitution. Solvents, temperatures, pressures, and other reaction conditions may readily be selected by one of ordinary skill in the art. Starting materials are commercially available or readily prepared by one of ordinary skill in the art. Constituents of compounds are as defined herein or elsewhere in the specification.

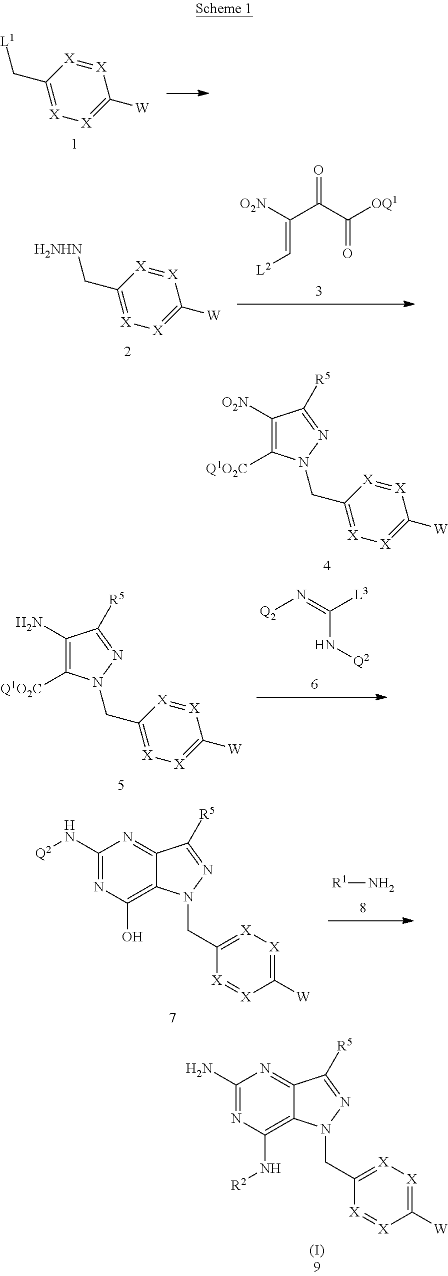

General routes to compounds described in the invention are illustrated in the Schemes, where the R1, R⁵, L¹, L², L³, Q¹, Q², X and W substituents are defined previously in the text or a functional group that can be converted to the desired final substituent. L is a leaving group such as a halide, OH that can be easily converted to a leaving group such as a triflate, thiother or heterocycle. As shown in Scheme 1, a general procedure for the preparation of compounds of the invention involves starting with a substituted benzyl derivative 1. Substitution of 1 with a suitably protected hydrazine using a suitable reagent can yield functionalized benyzl derivatives 2. For example, 2 could arise from a displacement reaction between a benzyl halide such as of methyl 4-(bromomethyl)-3-methoxybenzoate and a suitably protected hydrazine such as of tert-butyl hydrazinecarboxylate using one of many available base reagents, such as DIPEA or K₂CO₃, in a suitable solvent, such as DMF, followed by protecting group removal using standard conditions known in the literature. Subsequent reaction of 2 with a suitably substituted alkenoate 3 using conditions known to effect cyclization can provide the appropriately substituted nitropyrazole 4. For example, benzyl hydrazine 2 can undergo a cyclization reaction with methyl (Z)-4-(dimethylamino)-3-nitro-2-oxobut-3-enoate using a suitable base to provide nitropyrazole 4. Reduction of nitropyrazole 4 to aminopyrazole 5 can be accomplished using standard conditions known in the literature, such as H₂ (g) with Pd—C or Zn (s) with NH4OAc. Reaction of a suitably substituted 5 with an appropriately functionalized imidate 6 and cyclization of the resulting guandino intermediate under basic conditions, such as NaOMe-MeOH, can provide the hydroxypyrimidine 7. Coupling of 7 with an appropriately substituted amine 8 employing standard conditions known in the literature, followed by deprotection if necessary, provides compounds 9.

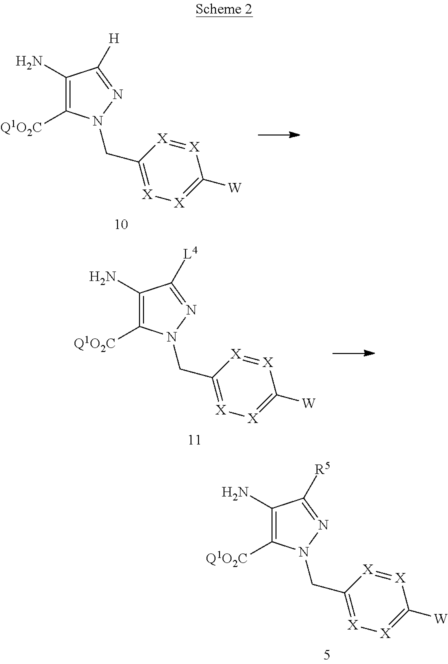

As illustrated in Scheme 2, the group at R5 may be manipulated to introduce substitutents prior to forming the pyrazolopyrimidine ring. A suitable leaving group L⁴ can be installed in aminopyrazole 10 in preparation for subsequent chemistry. For example, an installation of a halogen group can be accomplished using a suitable halogenating reagent such as NBS or NIS. Subsequent reaction of 11 using known carbon-carbon bond forming reactions such as Suzuki reactions or known carbon-heteroatom reactions such as Buchwald reactions under conditions described in the literature can be used to install alkyl, cycloalkyl, aryl or heteroaryl substituents at R⁵.

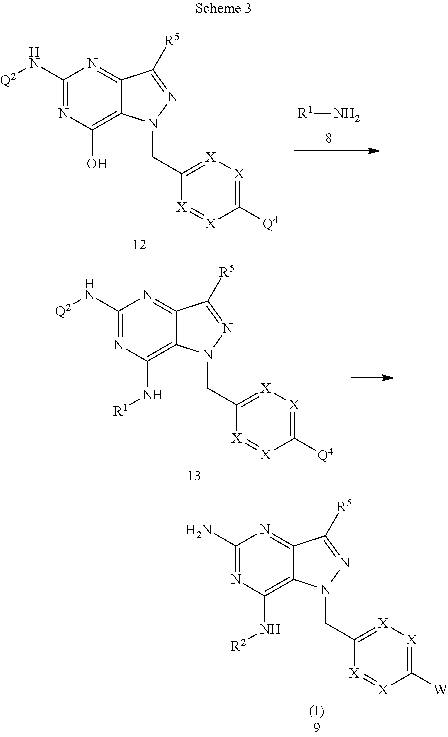

An alternate synthesis of pyrazolopyrimidine 9 are shown in Schemes 3 and 4. Using the synthetic routes described in Schemes 1 and 2, compound 12 can be prepared with a placeholder functional group at Q⁴. After coupling with amine 8 using standard literature conditions, Q⁴ can be transformed into W using a variety of means available to someone skilled in the art. For example, when Q⁴ is an ester, it can be reduced to the primary alcohol using standard conditions such as LiAlH₄ or LiBH₄, transformed into a suitable leaving group, such as —Cl, —Br or —OTs which can be displaced by a variety of nucleophiles. Deprotection, if necessary, then affords the pyrazolopyridimidine 9. In another variation, placeholder functional group Q⁴ as shown in Scheme 4, compound 12, can be transformed into W as in compound 14 in advance of coupling with amine 8.

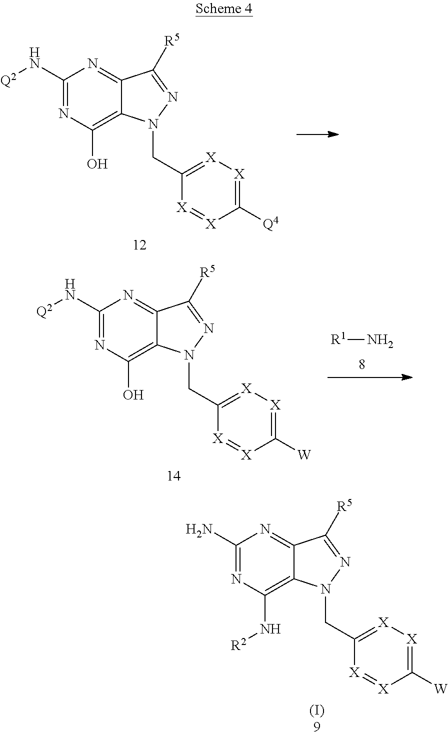

### Synthesis—Specific Examples

To further illustrate the foregoing, the following non-limiting, the following exemplary synthetic schemes are included. Variations of these examples within the scope of the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher is found in Table A.

### Example 1—Intermediate A

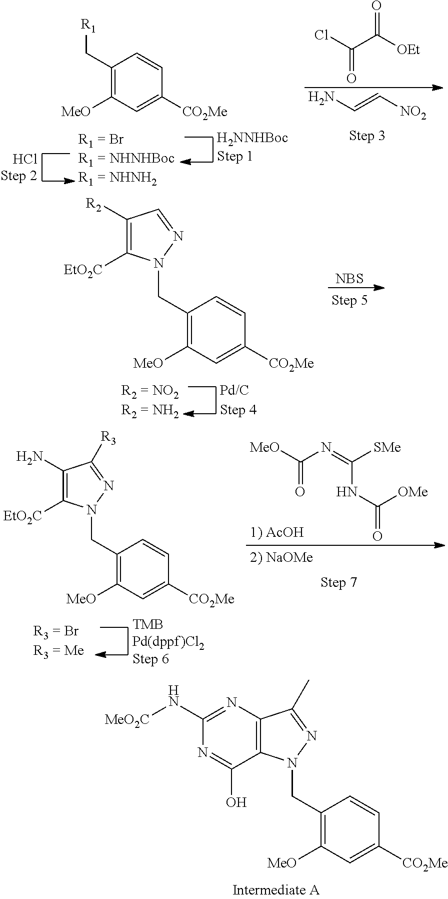

Intermediate A is useful for the synthesis of compounds of this disclosure.

Step 1: A solution of tert-butyl hydrazinecarboxylate (12.75 g, 96 mmol) and DIPEA in DMF (24 mL) at RT was treated with the dropwise addition of methyl 4-(bromomethyl)-3-methoxybenzoate (5 g, 19.30 mmol) in 24 mL of DMF via additional funnel over 1 h. The reaction mixture was stirred at RT overnight. EtOAc (135 mL) and H₂O (75 mL) were added and the biphasic mixture was stirred for 30 min. The reaction mixture was poured into a separatory funnel and the aqueous layer was removed. The organic layer was washed with 2 additional portions of H₂O (75 mL), 2 portions of 10% LiCl solution (75 mL), dried over Na₂SO₄ and concentrated. Column chromatography (Isco, 220 g SiO₂, 0% CH₂Cl₂ (5 min) then 15% EtOAc-CH₂Cl₂) provided tert-butyl 2-(2-methoxy-4-(methoxycarbonyl)benzyl)hydrazine-1-carboxylate as a clear oil (3.85 g).

¹H NMR (400 MHz, CHLOROFORM-d) δ 7.64 (dd, J=7.7, 1.5 Hz, 1H), 7.56 (d, J=1.5 Hz, 1H), 7.37 (d, J=7.7 Hz, 1H), 6.08-5.87 (m, 1H), 4.07 (s, 2H), 3.94 (d, J=4.6 Hz, 6H), 1.50-1.40 (m, 9H). LC/MS [M+H]⁺ 311.2; LC RT=0.80 min (Method A).

Step 2: tert-Butyl 2-(2-methoxy-4-(methoxycarbonyl)benzyl)hydrazine-1-carboxylate (25.4 g, 82 mmol) was dissolved in MeOH (164 mL) at RT. 4 N HCl-dioxane (123 ml, 59.5 mmol) was added and the reaction was stirred at RT overnight. The white precipitate was collected by filtration and dried to afford methyl 4-(hydrazineylmethyl)-3-methoxybenzoate, 2-HCl (20 g).

¹H NMR (400 MHz, DMSO-d6) δ 9.12 (br s), 7.62-7.55 (m, 1H), 7.53-7.47 (m, 2H), 4.10 (s, 2H), 3.88 (s, 3H), 3.87 (s, 3H).

LC/MS [M+H]⁺ 211.1; LC RT=0.51 min. (Method A)

Step 3: A solution of (E)-N,N-dimethyl-2-nitroethen-1-amine (46.4 g, 400 mmol) and pyridine (420 ml, 5195 mmol) in CH₂Cl₂ (799 ml) was cooled to −10° C. and slowly treated with ethyl 2-chloro-2-oxoacetate (51.4 ml, 460 mmol). The reaction mixture was allow to warm to 25° C. over 2 h and stirred overnight. The CH₂Cl₂ was removed by rotary evaporation and methyl 4-(hydrazineylmethyl)-3-methoxybenzoate dihydrochloride (31.7 g, 112 mmol) was added to the reaction mixture. The solution was stirred for 2 h at RT and the solvent was removed under vacuum. The residue was washed with water, 1N aqueous HCl solution and extracted with EtOAc (3×). The organic layers were dried over Na₂SO₄, and concentrated. The residue was dissolved in CH₂Cl₂, passed through a short silica gel column and recrystallized from ethanol to afford ethyl 1-(2-methoxy-4-(methoxycarbonyl)benzyl)-4-nitro-1H-pyrazole-5-carboxylate (29.4 g).

¹H NMR (400 MHz, CHLOROFORM-d) δ 8.06 (s, 1H), 7.64 (dd, J=7.9, 1.5 Hz, 1H), 7.56 (d, J=1.5 Hz, 1H), 7.13 (d, J=7.8 Hz, 1H), 5.53 (s, 2H), 4.45 (q, J=7.2 Hz, 2H), 3.94 (s, 3H), 3.88 (s, 3H), 1.37 (t, J=7.2 Hz, 3H).

LC/MS [M+Na]⁺386.0; LC RT=0.98 min (Method A).

Step 4: Ethyl 4-amino-1-(2-methoxy-4-(methoxycarbonyl)benzyl)-1H-pyrazole-5-carboxylate (3.04 g, 9.12 mmol, 86% yield) and Pd—C (1.131 g, 0.531 mmol) were suspended in EtOAc/MeOH (1:1) (152 mL). The reaction flask was evacuated under vacuum and purged with H₂ (3×) before stirring under balloon pressure of H₂ (g). After 5 h, the reaction mixture filtered through CELITE™, and fresh Pd—C (1.131 g, 0.531 mmol) was added. The reaction flask was evacuated under vacuum and purged with H₂ (3×) before stirring for 16 h under balloon pressure of H₂. The reaction mixture was filtered through CELITE™, concentrated and dried under vacuum to afford ethyl 4-amino-1-(2-methoxy-4-(methoxycarbonyl)benzyl)-1H-pyrazole-5-carboxylate (3.04 g) as a cream colored powder.

¹H NMR (400 MHz, DMSO-d6) δ 7.52-7.49 (m, 1H), 7.47 (dd, J=7.9, 1.5 Hz, 1H), 7.19 (s, 1H), 6.40 (d, J=7.8 Hz, 1H), 5.54 (s, 2H), 5.10 (s, 1H), 4.15 (q, J=7.1 Hz, 2H), 3.91 (s, 3H), 3.84 (s, 3H), 1.14 (t, J=7.1 Hz, 3H).

LC/MS [M+H]⁺ 334.1; LC/RT=0.85 min. (Method B).

Step 5: Ethyl 4-amino-1-(2-methoxy-4-(methoxycarbonyl)benzyl)-1H-pyrazole-5-carboxylate (1.65 g, 4.95 mmol) was dissolved in CHCl₃ (49.5 ml) and cooled to 0° C. NBS (0.925 g, 5.20 mmol) was added. After 15 min, the reaction was diluted with CHCl₃ and vigorously stirred with 10% aqueous sodium thiosulfate solution for 10 minutes. The organic phase was separated, washed with H₂O, dried over MgSO₄ and concentrated. The crude product was purified by column chromatography (80 g SiO₂, 0 to 50% EtOAc-hexane gradient elution) to afford ethyl 4-amino-3-bromo-1-(2-methoxy-4-(methoxycarbonyl)benzyl)-1H-pyrazole-5-carboxylate (1.32 g) as a white solid.

¹H NMR (400 MHz, DMSO-d6) δ 7.61-7.41 (m, 2H), 6.55 (d, J=8.3 Hz, 1H), 5.56 (s, 2H), 5.02 (s, 2H), 4.20 (q, J=7.1 Hz, 2H), 3.90 (s, 3H), 3.85 (s, 3H), 1.15 (t, J=7.1 Hz, 3H).

LC/MS [M+H]⁺ 412.2; LC RT=1.02 min (Method A).

Step 6: Ethyl 4-amino-3-bromo-1-(2-methoxy-4-(methoxycarbonyl)benzyl)-1H-pyrazole-5-carboxylate (741.2 mg, 67.1% yield), K₂CO₃ (1.098 g, 7.94 mmol) and TMB (3.5 M in THF) (1.816 ml, 6.36 mmol) were suspended in dioxane (26.5 ml):water (5.30 ml) (5:1). A stream of N₂ was bubbled through the reaction mixture for 5 min before the addition of PdCl₂(dppf)-CH₂Cl₂ adduct (0.052 g, 0.064 mmol). Stirring was continued for another 4 min before sealing the reaction flask and heating to 90° C. After 3 h, additional TMB (3.5 M in THF; 0.908 mL, 3.18 mmoL) and PdCl₂(dppf)-CH₂Cl₂ adduct (0.052 g, 0.064 mmol) were added. The reaction mixture was stirred at 100° C. for 16 h. The cooled reaction mixture was diluted with 100 mL of EtOAc and filtered through CELITE™, washing with additional EtOAc. The crude product was concentrated onto 4 g CELITE™. Column chromatography (80 g SiO₂, 0 to 30% EtOAc-CH₂Cl₂ gradient elution) afforded ethyl 4-amino-1-(2-methoxy-4-(methoxy-carbonyl)benzyl)-3-methyl-1H-pyrazole-5-carboxylate (741 mg) as a cream colored solid.

¹H NMR (400 MHz, DMSO-d6) δ 7.49 (d, J=1.5 Hz, 1H), 7.46 (dd, J=7.9, 1.5 Hz, 1H), 6.40 (d, J=7.8 Hz, 1H), 5.48 (s, 2H), 4.94-4.86 (m, 2H), 4.14 (q, J=7.0 Hz, 2H), 3.90 (s, 3H), 3.84 (s, 3H), 2.10 (s, 3H), 1.15-1.08 (m, 3H).

LC/MS [M+H]⁺ 348.2; LC/RT=0.89 min. (Method A).

Step 7: Ethyl 4-amino-1-(2-methoxy-4-(methoxycarbonyl)benzyl)-3-methyl-1H-pyrazole-5-carboxylate (742 mg, 2.136 mmol) was suspended in MeOH (10.800 mL) and heated gently with vigorous stirring to solubilize the material. 1,3-bis-(Methoxycarbonyl)-2-methyl-2-thiopseudourea (661 mg, 3.20 mmol), was added followed by AcOH (0.611 mL, 10.68 mmol). The reaction mixture was stirred at RT for 16 h. An additional portion of AcOH was added (0.049 mL, 0.854 mmol) and the reaction was stirred at RT for another 72 h before the addition of NaOMe (25% wt in MeOH) (5.69 mL, 25.6 mmol). After stirring for 3 h, the reaction mixture was re-acidified with AcOH. The product was collected by filtration, air-dried for 10 minutes and thoroughly dried in a chem-dry oven to afford methyl 4-((7-hydroxy-5-((methoxycarbonyl)-amino)-3-methyl-1H-pyrazolo[4,3-d]pyrimidin-1-yl)methyl)-3-methoxybenzoate (Intermediate A) (722.0 mg) as a cream colored solid.

¹H NMR (400 MHz, DMSO-d6) δ 11.58-11.17 (m, 2H), 7.51 (d, J=1.4 Hz, 1H), 7.49-7.42 (m, 1H), 6.67 (d, J=7.9 Hz, 1H), 5.67 (s, 2H), 3.90 (s, 3H), 3.84 (s, 3H), 3.71 (s, 3H), 2.31 (s, 3H).

LC/MS [M+H]⁺ 402.3; LC RT=0.86 min (Method A).

### Example 2—Compound 112

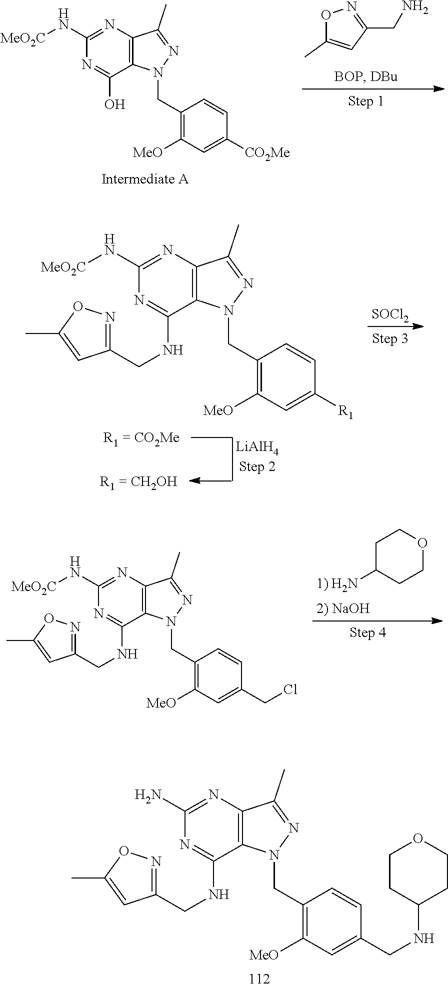

Step 1: A suspension of methyl 4-((7-hydroxy-5-((methoxycarbonyl)amino)-3-methyl-1H-pyrazolo[4,3-d]pyrimidin-1-yl)methyl)-3-methoxybenzoate (Intermediate A, 200 mg, 0.498 mmol) and BOP (331 mg, 0.747 mmol) in DMF (2491 μl) at RT was treated with (5-methyl-isoxazol-3-yl)methanamine (72.6 mg, 0.648 mmol) and DBU (3 eq) (225 μl, 1.495 mmol). The reaction mixture was heated to 40° C. After 15 min, additional DBU (2 eq.; 150 μL, 0.997 mmol) was added. The reaction mixture was stirred at 40° C. for 16 h. After cooling to RT, the reaction mixture was partitioned between EtOAc and half-saturated aqueous NaHCO₃. The organic phase was separated and the aqueous phase was extracted with EtOAc (2×). The combined organic layers were washed sequentially with 10% aqueous LiCl solution and brine, dried over Na₂SO₄ and concentrated. Column chromatography (12 g SiO₂, 0 to 10% CH₃OH—CH₂Cl₂ gradient elution) afforded methyl 3-methoxy-4-((5-((methoxycarbonyl)amino)-3-methyl-7-(((5-methyl-isoxazol-3-yl)methyl)amino)-1H-pyrazolo[4,3-d]pyrimidin-1-yl)methyl)benzoate (201.1 mg).

LC/MS [M+H]⁺ 496.2; LC RT=0.79 min (Method A).

Step 2: Methyl 3-methoxy-4-((5-((methoxycarbonyl)amino)-3-methyl-7-(((5-methylisoxazol-3-yl)methyl)amino)-1H-pyrazolo[4,3-d]pyrimidin-1-yl)methyl)benzoate (200 mg, 0.404 mmol) was suspended in THF at RT and sonicated to aid dissolution. LiAlH₄ (1M in THF; 807 μL, 0.807 mmol) was added dropwise over 10 min. After 20 min, the reaction was quenched with MeOH and partitioned between EtOAc and Rochelle salt. The biphasic mixture was stirred at RT for 2 h. The aqueous layer was separated and re-extracted with EtOAc (1×). The combined organic layers were washed with brine and concentrated. Column chromatography (12 g SiO₂, 0 to 10% CH₃OH—CH₂Cl₂ gradient elution) afforded methyl (1-(4-(hydroxymethyl)-2-methoxybenzyl)-3-methyl-7-(((5-methylisoxazol-3-yl)methyl)amino)-1H-pyrazolo[4,3-d]pyrimidin-5-yl)carbamate (73 mg).

LC/MS [M+H]⁺ 468.4; LC RT=0.62 min. (Method A).

Step 3: Methyl (1-(4-(hydroxymethyl)-2-methoxybenzyl)-3-methyl-7-(((5-methylisoxazol-3-yl)methyl)amino)-1H-pyrazolo[4,3-d]pyrimidin-5-yl)carbamate (73 mg, 0.156 mmol) was dissolved in CH₂Cl₂ (1562 μL) at RT. SOCl₂ (57.0 μl, 0.781 mmol) was added and the reaction stirred for 20 minutes. Concentration afforded methyl (1-(4-(chloromethyl)-2-methoxybenzyl)-3-methyl-7-(((5-methylisoxazol-3-yl)methyl)amino)-1H-pyrazolo[4,3-d]pyrimidin-5-yl)carbamate (80 mg) in sufficient purity to use without further purification.

LC/MS [M+H]⁺ 486.1; LC RT=0.83 min (Method A).

Step 4: A stock solution of methyl (1-(4-(chloromethyl)-2-methoxybenzyl)-3-methyl-7-(((5-methylisoxazol-3-yl)methyl)amino)-1H-pyrazolo[4,3-d]pyrimidin-5-yl)carbamate (20 mg, 0.041 mmol) in acetonitrile (412 μL) was treated with tetrahydro-2H-pyran-4-amine (12.49 mg, 0.123 mmol). The reaction was stirred at 40° C. overnight. After cooling to RT, the reaction mixture was concentrated, re-dissolved in dioxane (400 μL) and treated with 10 M NaOH (82 μL, 0.823 mmol). The reaction mixture was heated to 80° C. for 5 h. After cooling to RT, the reaction was neutralized with AcOH (42 μL) and concentrated. The crude product was dissolved in DMF, filtered through a PTFE frit and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation to afford Compound 112 (5.1 mg).

Compound 113 was analogously prepared: The crude product was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 24 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford Compound 113 (8.6 mg).

### Example 3—Compound 101

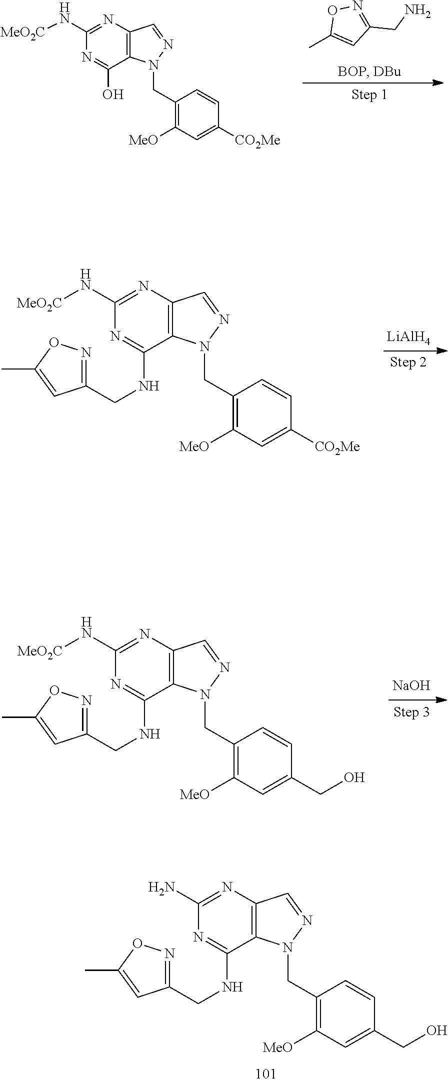

Step 1: A solution of methyl 4-((7-hydroxy-5-((methoxycarbonyl)amino)-1H-pyrazolo[4,3-d]pyrimidin-1-yl)methyl)-3-methoxybenzoate (US 2020/0038403 A1; 300 mg, 0.774 mmol) in DMSO (3.9 mL) was treated with (5-methylisoxazol-3-yl)methanamine (174 mg, 1.55 mmol), BOP (411 mg, 0.929 mmol) and DBU (233 μl, 1.549 mmol). The reaction mixture was stirred at RT for 2 h, diluted with EtOAc, and washed with H₂O (3×). The organic layer was dried over Na₂SO₄, filtered and concentrated in vacuo to give methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (353 mg, 95% yield).

¹H NMR (400 MHz, DMSO-d₆) δ 9.80 (s, 1H), 7.99-7.93 (m, 1H), 7.77 (t, J=5.9 Hz, 1H), 7.49 (d, J=1.5 Hz, 1H), 7.45 (dd, J=7.8, 1.5 Hz, 1H), 6.62 (d, J=7.9 Hz, 1H), 6.10 (d, J=0.9 Hz, 1H), 5.80 (s, 2H), 4.73 (d, J=5.9 Hz, 2H), 3.84 (s, 3H), 3.82 (s, 3H), 3.64 (s, 3H), 2.31 (s, 3H).

LC RT: 0.67 min. LC/MS [M+H]⁺ 482.3 (Method A)

Step 2: A solution of methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (190 mg, 0.395 mmol) in THF (10 mL) was cooled to 0° C. and treated with LiAlH₄ (1M in THF, 691 μL, 0.691 mmol). The reaction mixture was stirred for 15 min at 0° C., quenched with MeOH and Rochelle salt (saturated aqueous solution), and stirred at RT for 1 h. The mixture was extracted with EtOAc (3×). The combined organic layers were washed with H₂O, dried over Na₂SO₄, filtered and concentrated in vacuo to give methyl (1-(4-(hydroxymethyl)-2-methoxybenzyl)-7-(((5-methylisoxazol-3-yl)methyl)amino)-1H-pyrazolo[4,3-d]pyrimidin-5-yl)carbamate (160 mg, 89% yield).

¹H NMR (400 MHz, DMSO-d6) δ 9.77-9.75 (m, 1H), 7.90-7.88 (m, 1H), 7.72 (br t, J=5.7 Hz, 1H), 6.94 (s, 1H), 6.76 (d, J=7.5 Hz, 1H), 6.61-6.57 (m, 1H), 6.15 (d, J=0.8 Hz, 1H), 5.68 (s, 2H), 5.16 (t, J=5.7 Hz, 1H), 4.73 (br d, J=5.8 Hz, 2H), 4.44 (d, J=5.6 Hz, 2H), 3.70 (s, 3H), 3.62 (s, 3H), 2.33 (s, 3H).

LC RT: 0.58 min. LCMS [M+H]⁺=454.3 (Method A)

Step 3: A solution of methyl (1-(4-(hydroxymethyl)-2-methoxybenzyl)-7-(((5-methylisoxazol-3-yl)methyl)amino)-1H-pyrazolo[4,3-d]pyrimidin-5-yl)carbamate (22 mg, 0.048 mmol) in Dioxane (500 μL) was treated with NaOH (10 M aqueous solution, 200 μL, 2.0 mmol) and heated to 75° C. After 5 h, the reaction mixture was cooled to RT, neutralized with HOAc (114 μL, 2.0 mmol) and concentrated under a stream of nitrogen. The residue was dissolved in DMF and filtered through a PTFE frit. The crude material was purified via preparative LC/MS with the following conditions: Column: XBridge C18, 200 mm×19 mm, 5-μm particles; Mobile Phase A: 5:95 acetonitrile:water with 10 mM NH₄OAc; Mobile Phase B: 95:5 acetonitrile: water with 10 mM NH₄OAc; Gradient: a 0-minute hold at 9% B, 9-49% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 101 (3.5 mg, 8% yield).

### Example 4—Compound 102

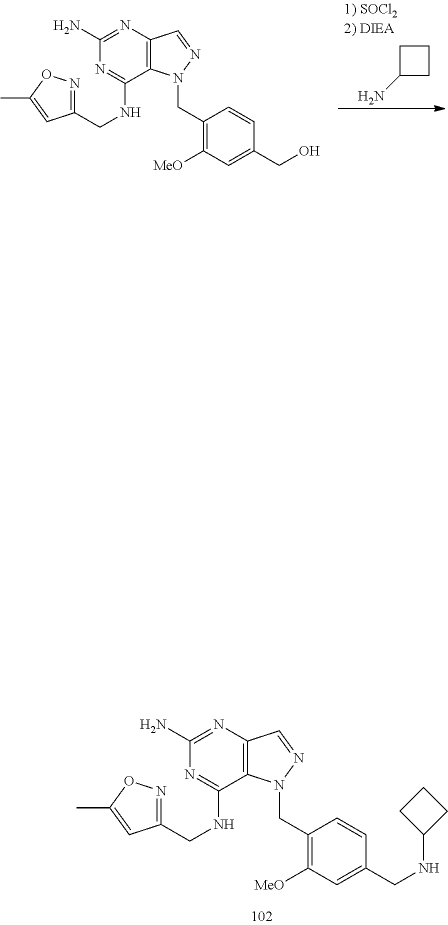 [SMILES: COc1cc(CO)ccc1Cn1ncc2nc(N)nc(NCc3cc(C)on3)c21 ; NC1CCC1 ; COc1cc(CNC2CCC2)ccc1Cn1ncc2nc(N)nc(NCc3cc(C)on3)c21]

SOCl₂ (24 μL, 0.33 mmol) was added to a RT solution of (4-((5-amino-7-(((5-methylisoxazol-3-yl)methyl)amino)-1H-pyrazolo[4,3-d]pyrimidin-1-yl)methyl)-3-methoxy-phenyl)methanol (26.3 mg, 0.067 mmol) in THF (0.7 mL). After stirring for 30 min, the reaction mixture was concentrated in vacuo. The residue was re-dissolved in DCM and concentrated in vacuo. The residue was dissolved in DMF (0.7 mL) treated with cyclobutanamine (25.3 mg, 0.355 mmol) and stirred at RT for 3 h. The temperature was raised to 70° C. The reaction mixture was stirred for an additional 2 h and concentrated in vacuo. The crude product was dissolved in DMF and filtered through a PTFE frit. The crude material was purified via preparative LC/MS with the following conditions: Column: XBridge C18, 200 mm×19 mm, 5-μm particles; Mobile Phase A: 5:95 acetonitrile:water with 10 mM NH₄OAc; Mobile Phase B: 95:5 acetonitrile: water with 10 mM NH₄OAc; Gradient: a 0-minute hold at 2% B, 2-42% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give a residue which was further purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 22 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 102 as the bis TFA salt (4.0 mg, 11%).

### Example 5—Compound 103

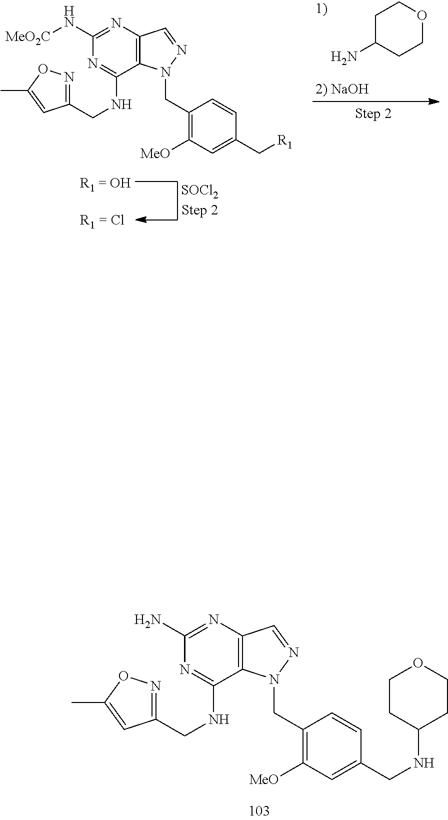

Step 1: A solution of methyl (1-(4-(hydroxymethyl)-2-methoxybenzyl)-7-(((5-methylisoxazol-3-yl)methyl)amino)-1H-pyrazolo[4,3-d]pyrimidin-5-yl)carbamate (159 mg, 0.35 mmol) in DCM (3.5 mL) was treated with SOCl₂ (128 μL, 1.76 mmol). The reaction mixture was stirred at RT for 15 min and concentrated in vacuo. The residue was re-dissolved in DCM and concentrated in vacuo to give methyl (1-(4-(chloromethyl)-2-methoxybenzyl)-7-(((5-methyl-isoxazol-3-yl)methyl)amino)-1H-pyrazolo[4,3-d]pyrimidin-5-yl)carbamate (182 mg, 100%).

LC RT: 0.80 min. LCMS [M+H]⁺=472.3 (Method A)

Step 2: A solution of methyl (1-(4-(chloromethyl)-2-methoxybenzyl)-7-(((5-methylisoxazol-3-yl)methyl)amino)-1H-pyrazolo[4,3-d]pyrimidin-5-yl)carbamate (25 mg, 0.053 mmol in DMF (1.1 mL) was treated with tetrahydro-2H-pyran-4-amine (26.8 mg, 0.265 mmol). The reaction mixture was stirred at 70° C. for 2 h and concentrated in vacuo. The residue was re-dissolved in dioxane (0.5 mL) at RT, treated with NaOH (10M aqueous solution, 27 μl, 0.27 mmol) and heated to 80° C. for 4.5 h. The reaction mixture was neutralized at RT with HOAc (15 μl, 0.27 mmol) and concentrated in vacuo. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-30% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation to give Compound 103 as the bis TFA salt (20.2 mg, 54%).

The following compounds were analogously prepared: Compound 104, Compound 105, Compound 106, Compound 110, and Compound 111.

### Example 6—Compound 107

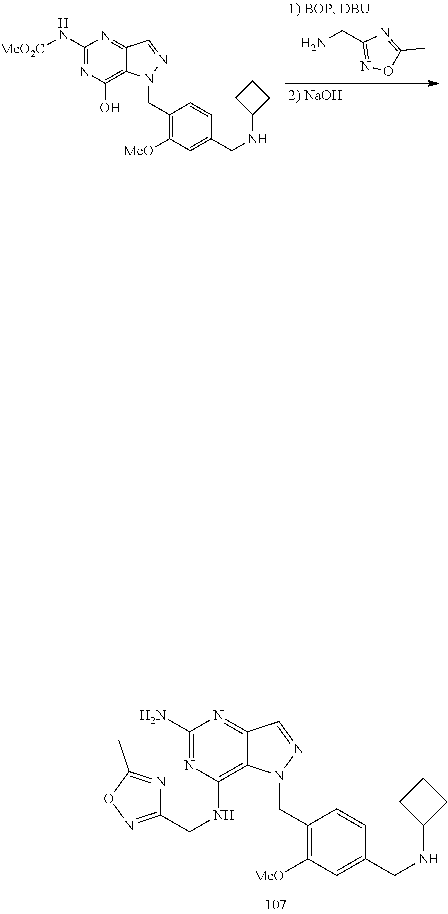

A solution of methyl (1-(4-((cyclobutylamino)methyl)-2-methoxybenzyl)-7-hydroxy-1H-pyrazolo[4,3-d]pyrimidin-5-yl)carbamate (US 2020/0038403 A1; 30 mg, 0.073 mmol) in DMF (0.7 mL) was treated with BOP (57.9 mg, 0.131 mmol), (5-methyl-1,2,4-oxadiazol-3-yl)methan-amine-HCl (54.4 mg, 0.364 mmol) and DBU (164 μL, 1.091 mmol). The reaction mixture was stirred at RT for 2 h, diluted with EtOAc, and washed with saturated NaHCO₃ solution and H₂O. The organic layer was concentrated in vacuo. The residue was dissolved in dioxane (0.7 mL), treated with NaOH (10 M aqueous solution, 0.20 mL, 2.0 mmol), and heated to 75° C. After 4 h, the reaction mixture was cooled to RT, neutralized with HOAc (0.12 mL, 2.0 mmol) and concentrated in vacuo. The crude product was dissolved in DMF and H₂O, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 107 (8.6 mg, 26% yield).

### Example 7—Compound 114

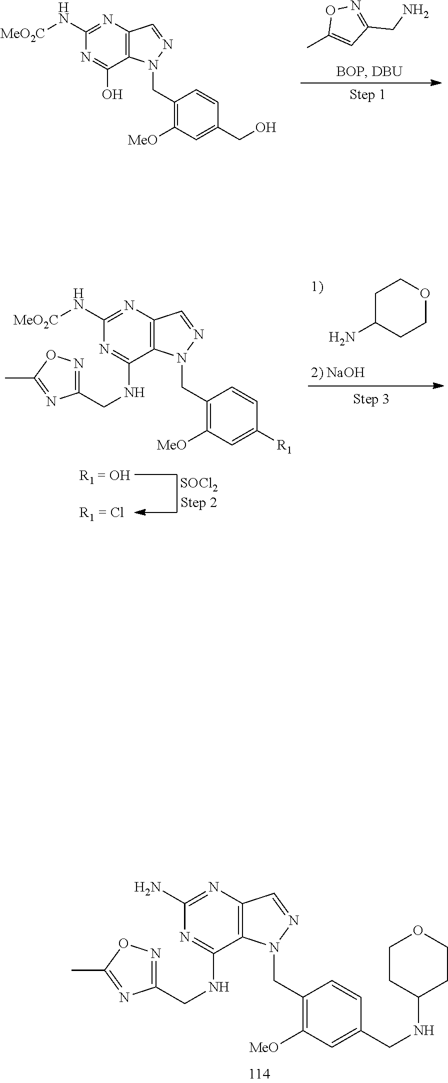

Step 1: A solution of methyl (7-hydroxy-1-(4-(hydroxymethyl)-2-methoxybenzyl)-1H-pyrazolo[4,3-d]pyrimidin-5-yl)carbamate (US 2020/0038403 A1, FIG. 7, compound 64; 700 mg, 1.95 mmol) in DMSO (9.7 mL) was treated with (5-methyl-1,2,4-oxadiazol-3-yl)methan-amine-HCl (379 mg, 2.53 mmol), BOP (129 mg, 2.92 mmol) and DBU (1.0 mL, 6.8 mmol). The reaction mixture was stirred at RT for 2 h, diluted with DCM, and washed with H₂O. The organic layer was washed with H₂O (6×), dried over Na₂SO₄, filtered, and concentrated in vacuo. The residue was dissolved in DCM/MeOH, absorbed onto CELITE™ and purified via column chromatography (100 g C₁₈ gold column; Mobile Phase A: 5:95 acetonitrile:water with 0.05% TFA; Mobile Phase B: 95:5 acetonitrile:water with 0.05% TFA; Flow Rate: 60 mL/min, 10-50% gradient). The purified product was dissolved in DCM and washed with saturated aqueous NaHCO₃ solution. The organic layer was dried over Na₂SO₄, filtered and concentrated in vacuo to give methyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (372 mg, 42% yield).

¹H NMR (400 MHz, DMSO-d₆) δ 9.69-9.66 (m, 1H), 7.89 (s, 1H), 7.76 (t, J=5.8 Hz, 1H), 6.95 (s, 1H), 6.81-6.77 (m, 1H), 6.76-6.70 (m, 1H), 5.69 (s, 2H), 5.17 (t, J=5.7 Hz, 1H), 4.89 (d, J=5.7 Hz, 2H), 4.45 (d, J=5.8 Hz, 2H), 3.77 (s, 3H), 3.60 (s, 3H), 2.56 (s, 3H).

LC RT: 0.56 min. LC/MS [M+H]⁺ 455.3 (Method A)

Step 2: A solution of methyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (372 mg, 0.818 mmol) in DCM (8.2 mL) was treated with SOCl₂ (179 μL, 2.46 mmol). The reaction mixture was stirred at RT for 10 min and concentrated in vacuo. The residue was re-dissolved in DCM and concentrated in vacuo to give methyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (387 mg, 100%).

¹H NMR (400 MHz, DMSO-d₆) δ 11.82-11.60 (m, 1H), 9.40-9.21 (m, 1H), 8.12-8.08 (m, 1H), 7.10 (s, 1H), 7.04-6.95 (m, 2H), 5.81 (s, 2H), 5.02 (br d, J=5.3 Hz, 2H), 4.74 (s, 2H), 3.80 (s, 3H), 3.75 (s, 3H), 2.60 (s, 3H).

LC RT: 0.70 min. LCMS [M+H]⁺=473.3 (Method A)

Step 3: A solution of methyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (34.7 mg, 0.073 mmol) in DMF (1.5 mL) was treated with tetrahydro-2H-pyran-4-amine (37.1 mg, 0.367 mmol). The reaction was stirred at 75° C. for 1 h and concentrated in vacuo. The residue was dissolved in dioxane (1.0 mL) and MeOH (0.2 mL), treated with NaOH (10M aqueous solution, 0.2 mL, 2.0 mmol) and heated at 75° C. for 2 h. After cooling to RT, the reaction mixture was neutralized with HOAc (0.12 mL, 2.0 mmol) and concentrated in vacuo. The crude product was dissolved in DMF and H₂O, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 0% B, 0-40% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 114 (7.5 mg, 18%).

The following compounds were analogously prepared: Compound 115, Compound 117, Compound 120, Compound 121, Compound 122, and Compound 123.

### Example 8—Compound 116

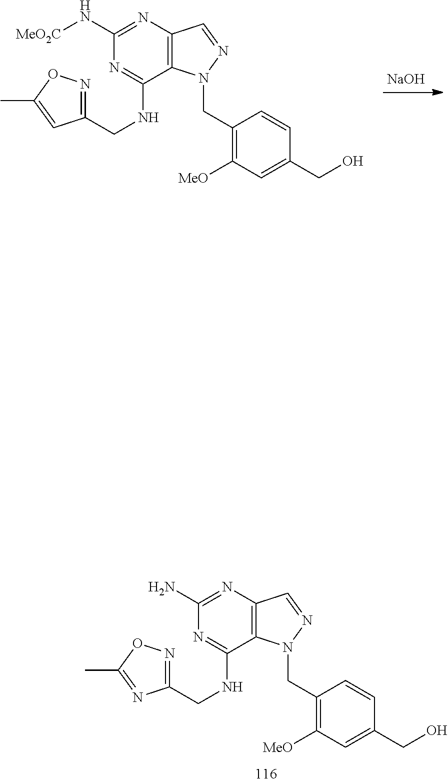 [SMILES: COC(=O)Nc1nc(NCc2cc(C)on2)c2c(cnn2Cc2ccc(CO)cc2OC)n1 ; "NaOH" ; COc1cc(CO)ccc1Cn1ncc2nc(N)nc(NCc3noc(C)n3)c21]

A solution of methyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (19 mg, 0.043 mmol) in Dioxane (0.4 mL) and MeOH (0.2 mL) was treated with NaOH (10 M aq solution, 50 μL, 0.5 mmol) and heated to 50° C. After 30 min, the reaction mixture was cooled to RT, neutralized with HOAc (30 μL, 0.5 mmol) and concentrated in vacuo. The residue was dissolved in DMF and filtered through a PTFE frit. The crude material was purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 2% B, 2-42% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 116 (3.9 mg, 22% yield).

### Example 9—Compound 109a

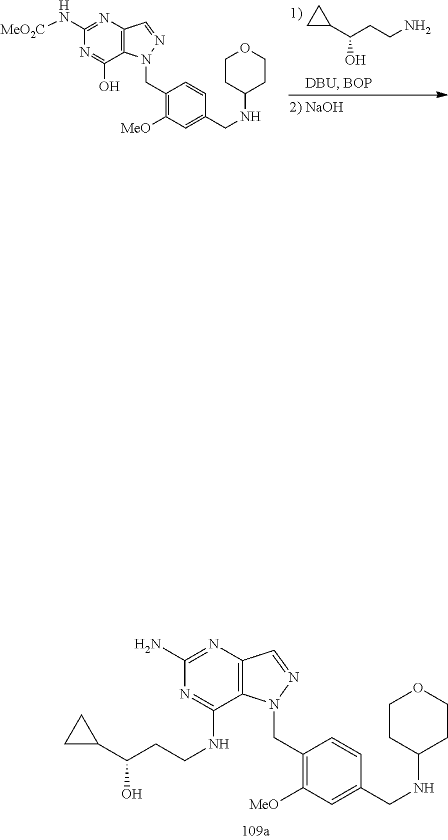

To a solution of methyl (7-hydroxy-1-(2-methoxy-4-(((tetrahydro-2H-pyran-4-yl)amino)methyl)benzyl)-1H-pyrazolo[4,3-d]pyrimidin-5-yl)carbamate (75 mg, 0.170 mmol, US 2020/0038403 A1) in DMSO (1.5 mL) was added (S)-3-amino-1-cyclopropylpropan-1-ol (39.0 mg, 0.339 mmol), DBU (0.077 mL, 0.509 mmol), and BOP (150 mg, 0.339 mmol); The reaction mixture was heated at 70° C. for 2 h, treated with 5M NaOH (0.136 mL, 0.678 mmol), and heated at 70° C. for 2 h. The reaction mixture was cooled to 25° C. and the crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 25 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 1% B, 1-41% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to afford Compound 109a (2.3 mg, 4.69 μmol, 2.77% yield).

Compound 109b was analogously prepared.

### Example 10—Compound 108

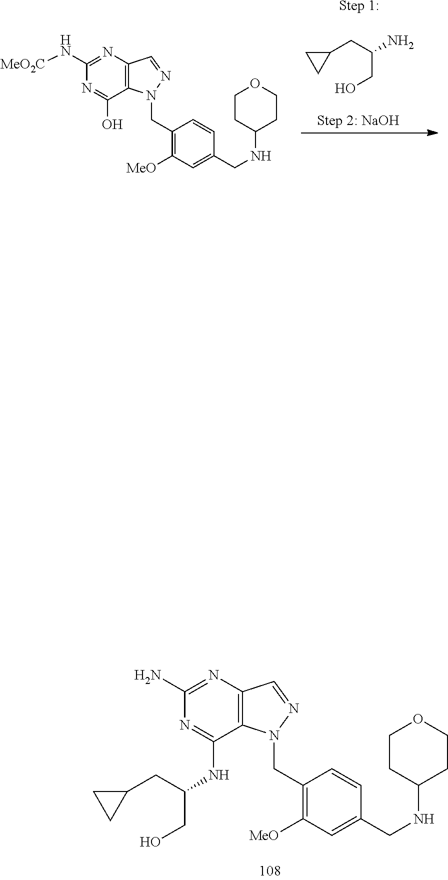

Step 1. To a solution of methyl (7-hydroxy-1-(2-methoxy-4-(((tetrahydro-2H-pyran-4-yl)amino)methyl)benzyl)-1H-pyrazolo[4,3-d]pyrimidin-5-yl)carbamate (90 mg, 0.203 mmol, US 2020/0038403 A1), (S)-2-Amino-3-cyclopropylpropan-1-ol hydrochloride (93 mg, 0.610 mmol) and BOP (135 mg, 0.305 mmol) in DMF (2034 μl) was added DBU (153 μl, 1.017 mmol). The reaction mixture was at RT overnight, diluted with water (2 mL, 0.2% TFA), and purified on Accq Prep 20×150 mm Xbridge column (6 injections): 20% acetonitrile/water (0.1% TFA) fractions collected at 12 min were lyophilyzed to provide methyl (S)-(7-((1-cyclopropyl-3-hydroxypropan-2-yl)amino)-1-(2-methoxy-4-(((tetrahydro-2H-pyran-4-yl)amino)methyl)benzyl)-1H-pyrazolo[4,3-d]pyrimidin-5-yl)carbamate (65 mg, 59.2% yield) as a white solid. LCMS [M+H]⁺=539.3.

Step 2. Methyl (S)-(7-((1-cyclopropyl-3-hydroxypropan-2-yl)amino)-1-(2-methoxy-4-(((tetrahydro-2H-pyran-4-yl)amino)methyl)benzyl)-1H-pyrazolo[4,3-d]pyrimidin-5-yl)carbamate (167 mg, 0.309 mmol) was dissolved in dioxane (5158 l) and treated with NaOH (619 μl, 3.09 mmol) and heated at 80° C. overnight. The reaction mixture was neutralized with HCl and concentrated. The residue was dissolved in DMF (4 mL) and filtered. The crude material was purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation to give Compound 108 (60 mg, 40% yield).

Compound 125 was analogously prepared.

### Example 11—Compound 126

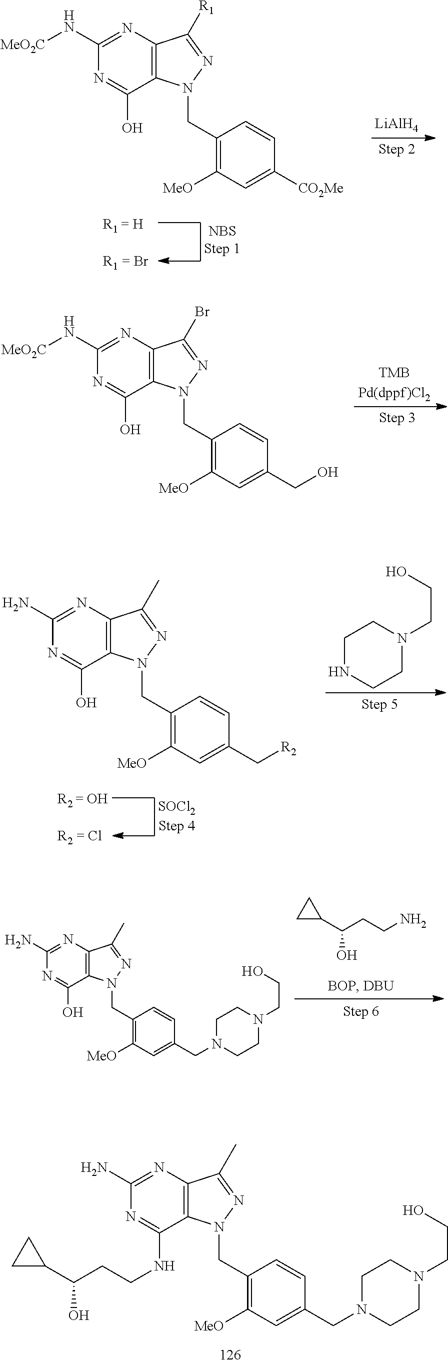

Step 1. To methyl 4-((7-hydroxy-5-((methoxycarbonyl)amino)-1H-pyrazolo[4,3-d]pyrimidin-1-yl)methyl)-3-methoxybenzoate (50 mg, 0.129 mmol) in DMF (1 mL) was added NBS (76 mg, 0.427 mmol). The reaction mixture was stirred at 40° C. overnight, cooled to 25° C., diluted with MeOH, and filtered to afford methyl 4-((3-bromo-7-hydroxy-5-((methoxycarbonyl)-amino)-1H-pyrazolo[4,3-d]pyrimidin-1-yl)methyl)-3-methoxybenzoate (40 mg, 0.082 mmol, 63.1% yield).

LC-MS m/z 468.2 [M+2H]⁺.

¹H NMR (400 MHz, DMSO-d₆) δ 11.86-11.17 (m, 2H), 7.51 (s, 2H), 7.02-6.74 (m, 1H), 5.74 (s, 2H), 3.86 (d, J=9.7 Hz, 6H), 3.76 (s, 3H)

Step 2. LiAlH₄ (1M in THF; 6 mL, 6.00 mmol) was added slowly to a solution of methyl 4-((3-bromo-7-hydroxy-5-((methoxycarbonyl)amino)-1H-pyrazolo[4,3-d]pyrimidin-1-yl)methyl)-3-methoxybenzoate (1 g, 2.145 mmol) in THF (20 mL) at 0° C. (ice bath). The reaction mixture was stirred at RT for 30 min. The reaction was quenched by slow addition of saturated Na₂SO₄ (5.0 ml) at 0° C. (ice bath). The mixture was stirred at RT for 30 min. The organic solvent removed on a rotary evaporator and the aqueous phase was lyophilized. The lyophilized material was diluted with MeOH (100 ml) and filtered (wash with 3×10 mL MeOH). The solvent was removed and the material purified on silica gel (DCM-MeOH 0-30%) to afford methyl (3-bromo-7-hydroxy-1-(4-(hydroxymethyl)-2-methoxybenzyl)-1H-pyrazolo[4,3-d]pyrimidin-5-yl)carbamate (330 mg, 0.753 mmol, 30% yield).

LC-MS m/z 440.2[M+2H]⁺.

¹H NMR (400 MHz, DMSO-d₆) δ 7.05-6.95 (m, 1H), 6.87-6.76 (m, 2H), 5.66 (s, 2H), 5.23-5.14 (m, 1H), 4.52-4.43 (m, 2H), 3.82-3.72 (m, 6H)

Step 3. A microwave vial was charged with methyl (3-bromo-7-hydroxy-1-(4-(hydroxymethyl)-2-methoxybenzyl)-1H-pyrazolo[4,3-d]pyrimidin-5-yl)carbamate (200 mg, 0.456 mmol) (ca. 80% pure contaminated with the N2-regioisomer), TMB (0.255 ml, 1.825 mmol), [1,1′-bis(diphenylphosphino)ferrocene]dichloropalladium(II) (100 mg, 0.137 mmol), K₂CO₃ (442 mg, 3.19 mmol), dioxane (8 mL) and water (2 mL). The reaction mixture was heated in a microwave oven at 120° C. for 1 hour, diluted with EtOAc, washed with water, and dried over Na₂SO₄. The solvent was removed and the material was purified on silica gel (dry load) DCM-MeOH 0-50% to afford 5-amino-1-(4-(hydroxymethyl)-2-methoxybenzyl)-3-methyl-1H-pyrazolo[4,3-d]pyrimidin-7-ol (49 mg, 0.093 mmol, 20.43% yield).

LC-MS m/z 316.3[M+H]⁺.

Step 4. To a 20 mL vial was added 5-amino-1-(4-(hydroxymethyl)-2-methoxybenzyl)-3-methyl-1H-pyrazolo[4,3-d]pyrimidin-7-ol (50 mg, 0.159 mmol) and DCM (2 mL) followed by the RT addition of SOCl₂ (0.1 mL, 1.370 mmol). The reaction mixture was stirred at 25° C. and concentrated in vacuo to give 5-amino-1-(4-(chloromethyl)-2-methoxybenzyl)-3-methyl-1H-pyrazolo[4,3-d]pyrimidin-7-ol (52.9 mg, 0.158 mmol, 100% yield), used without purification.

LC-MS m/z 335.7[M+2H]⁺.

Step 5. To 5-amino-1-(4-(chloromethyl)-2-methoxybenzyl)-3-methyl-1H-pyrazolo-[4,3-d]pyrimidin-7-ol (52 mg, 0.156 mmol) in DMF (2 mL) was added 2-(piperazin-1-yl)ethan-1-ol (0.1 mL, 0.815 mmol) The reaction mixture was stirred at 25° C. overnight and the solvent was removed. The material was purified on silica gel (dry load) DCM-MeOH 0-30% to afford 5-amino-1-(4-((4-(2-hydroxyethyl)piperazin-1-yl)methyl)-2-methoxybenzyl)-3-methyl-1H-pyrazolo[4,3-d]pyrimidin-7-ol (53 mg, 0.095 mmol, 61.3% yield).

LC-MS m/z 428.3[M+H]⁺.

Step 6. To a solution of 5-amino-1-(4-((4-(2-hydroxyethyl)piperazin-1-yl)methyl)-2-methoxybenzyl)-3-methyl-1H-pyrazolo[4,3-d]pyrimidin-7-ol (53 mg, 0.124 mmol) and (S)-3-amino-1-cyclopropylpropan-1-ol (30 mg, 0.260 mmol) in DMSO (1.5 mL) was added DBU (0.075 mL, 0.496 mmol) and BOP (110 mg, 0.248 mmol). The reaction mixture was heated at 70° C. for 1 h. The product was purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-min hold at 0% B, 0-40% B over 20 min, then a 0-min hold at 30 100% B; Flow Rate: 20 mL/min; Column Temperature: 25° C. Fraction collection was triggered by MS and UV signals. Fractions containing the desired product were combined and dried via centrifugal evaporation to yield Compound 126.

### Example 12—Compound 118

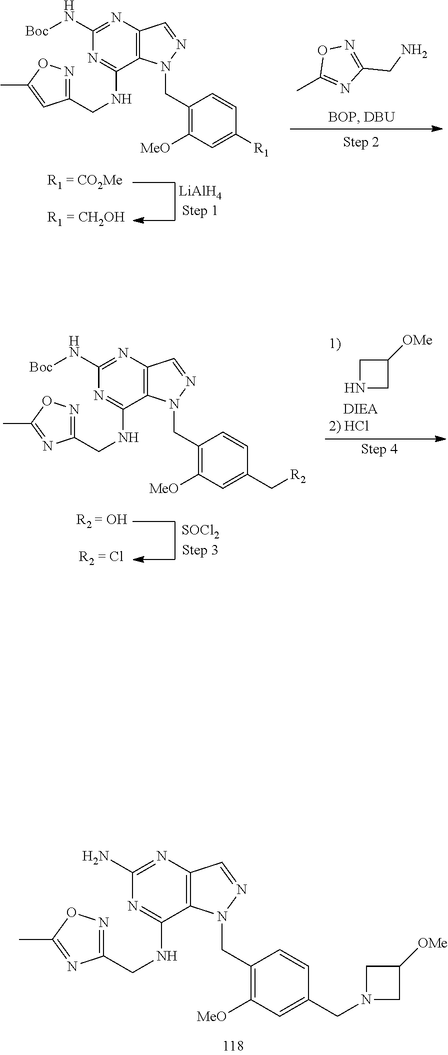

Step 1. A solution of methyl 4-((5-((tert-butoxycarbonyl)amino)-7-hydroxy-1H-pyrazolo[4,3-d]pyrimidin-1-yl)methyl)-3-methoxybenzoate (685 mg, 1.59 mmol; US 2020/0038403; FIG. 8, compound 71) in THF (16 mL) was cooled to 0° C. and treated with LiAlH₄ (1 M in THF, 2.8 mL, 2.8 mmol). The reaction mixture was stirred for 15 min at 0° C., quenched with H₂O and Rochelle salt (saturated aqueous solution) and stirred at RT for 3 h. The organic layer was absorbed onto CELITE™ and purified via column chromatography (24 g SiO₂; 0 to 20% MeOH-DCM gradient elution) to give tert-butyl (7-hydroxy-1-(4-(hydroxymethyl)-2-methoxybenzyl)-1H-pyrazolo[4,3-d]pyrimidin-5-yl)carbamate (460 mg, 72% yield). 1H (400 MHz, DMSO-d₆) δ 11.69-11.43 (m, 1H), 10.95-10.62 (m, 1H), 7.87-7.79 (m, 1H), 6.97 (s, 1H), 6.77 (d, J=7.7 Hz, 1H), 6.59 (d, J=7.8 Hz, 1H), 5.66 (s, 2H), 5.16 (t, J=5.8 Hz, 1H), 4.45 (d, J=5.8 Hz, 2H), 3.79 (s, 3H), 1.49 (s, 9H).

LC RT: 0.77 min. LC/MS [M+H]⁺=402.2 (Method D)

Step 2. A solution of tert-butyl (7-hydroxy-1-(4-(hydroxymethyl)-2-methoxybenzyl)-1H-pyrazolo[4,3-d]pyrimidin-5-yl)carbamate (460 mg, 1.15 mmol) in DMSO (5.7 mL) was treated with (5-methyl-1,2,4-oxadiazol-3-yl)methanamine-HCl (223 mg, 1.49 mmol), BOP (760 mg, 1.72 mmol) and DBU (0.69 mL, 4.6 mmol). The reaction mixture was stirred at RT for 2 h, diluted with EtOAc and washed with H₂O (2×). The organic layer was absorbed onto CELITE™ and purified via column chromatography (100 g C₁₈ gold column; Mobile Phase A: 5:95 acetonitrile:water with 0.05% TFA; Mobile Phase B: 95:5 acetonitrile:water with 0.05% TFA; Flow Rate: 60 mL/min, 30-50% gradient). The purified product was dissolved in DCM and washed with saturated aqueous NaHCO₃ solution. The organic layer was dried over Na₂SO₄, filtered and concentrated in vacuo to give tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (190 mg, 33% yield).

¹H NMR (400 MHz, DMSO-d₆) δ 9.24-9.15 (m, 1H), 7.87 (s, 1H), 7.72 (t, J=5.8 Hz, 1H), 6.95 (s, 1H), 6.82-6.75 (m, 1H), 6.73-6.68 (m, 1H), 5.68 (s, 2H), 5.17 (t, J=5.7 Hz, 1H), 4.87 (d, J=5.7 Hz, 2H), 4.44 (d, J=5.7 Hz, 2H), 3.76 (s, 3H), 2.55 (s, 3H), 1.43 (s, 9H).

LC RT: 0.75 min. LC/MS [M+H]⁺=497.2 (Method D)

Step 3. A solution of tert-butyl (1-(4-(hydroxymethyl)-2-methoxybenzyl)-7-(((5-methyl-1,2,4-oxadiazol-3-yl)methyl)amino)-1H-pyrazolo[4,3-d]pyrimidin-5-yl)carbamate (161 mg, 0.320 mmol) in DCM (0.65 mL) was treated with SOCl₂ (71 μL, 0.97 mmol). The reaction mixture was stirred at RT for 15 min and concentrated in vacuo. The residue was dissolved in DCM and concentrated in vacuo to give tert-butyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (166 mg, 100%).

LC RT: 0.89 min. LC/MS [M+H]⁺=515.2 (Method D)

Step 4. A solution of tert-butyl (1-(4-(chloromethyl)-2-methoxybenzyl)-7-(((5-methyl-1,2,4-oxadiazol-3-yl)methyl)amino)-1H-pyrazolo[4,3-d]pyrimidin-5-yl)carbamate (33 mg, 0.064 mmol) in DMF (1.3 mL) was treated with DIEA (113 μL, 0.645 mmol) and 3-methoxy-azetidine-HCl (23.9 mg, 0.193 mmol). The reaction mixture was stirred at 70° C. for 1 h and dried under N₂ stream, followed by further drying in vacuo. The residue was dissolved in dioxane (0.6 mL) and treated with HCl (4 M in dioxane, 0.82 mL, 3.3 mmol), stirred at 40° C. for 30 min and concentrated. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile:water with 10 mM NH₄OAc; Gradient: a 0-minute hold at 2% B, 2-42% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The isolated product was purified further via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-30% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 118 (9.4 mg, 21%).

Compound 119 was analogously prepared.

### Example 13—Compound 127

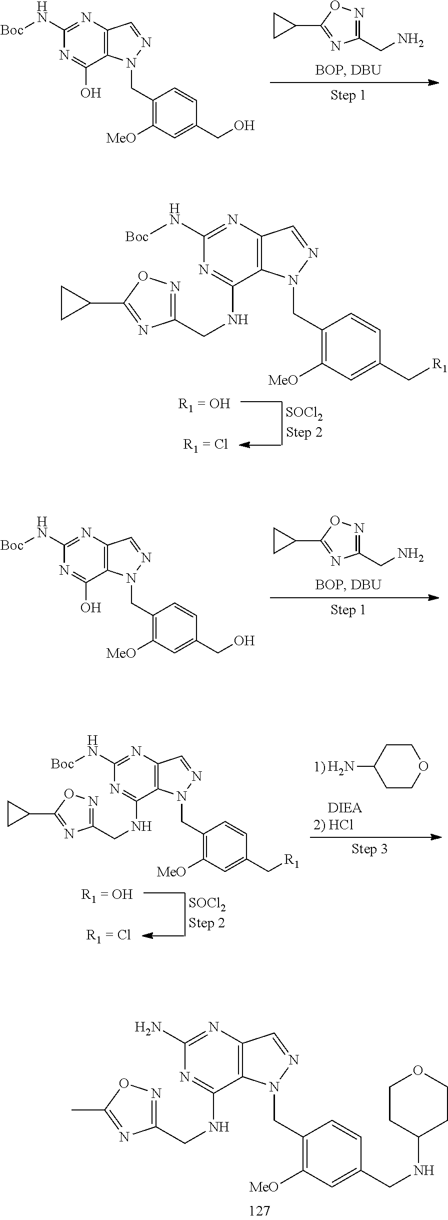

Step 1. A solution of tert-butyl (7-hydroxy-1-(4-(hydroxymethyl)-2-methoxybenzyl)-1H-pyrazolo[4,3-d]pyrimidin-5-yl)carbamate (200 mg, 0.498 mmol) in DMSO (2.5 mL) was treated with (5-cyclopropyl-1,2,4-oxadiazol-3-yl)methanamine-HCl (175 mg, 0.996 mmol), BOP (331 mg, 0.747 mmol) and DBU (0.30 mL, 2.0 mmol). The reaction mixture was stirred at RT for 2 h, diluted with EtOAc, and washed with H₂O (2×). The organic layer was concentrated in vacuo. The crude product was dissolved in MeOH, filtered through a PTFE frit, and purified via preparative HPLC with the following conditions: Column: Axia C18 100 mm×30 mm, 5-μm particles; Mobile Phase A: 10:90 Methanol: water with 0.1% TFA; Mobile Phase B: 90:10 MeOH: water with 0.1% TFA; Gradient: a 0-minute hold at 40% B, 40-55% B over 10 minutes, then a 5-minute hold at 55% B; Flow Rate: 40 mL/min; UV detection at 220 nm; Column Temperature: 25° C. The purified product was neutralized with saturated aqueous NaHCO₃ solution and washed with DCM. The organic layer was dried over Na₂SO₄, filtered and concentrated in vacuo to give tert-butyl (7-(((5-cyclopropyl-1,2,4-oxadiazol-3-yl)methyl)amino)-1-(4-(hydroxymethyl)-2-methoxybenzyl)-1H-pyrazolo[4,3-d]pyrimidin-5-yl)carbamate (93.2 mg, 36% yield).

¹H NMR (400 MHz, DMSO-d₆) δ 9.25-9.17 (m, 1H), 7.88 (s, 1H), 7.71 (t, J=5.7 Hz, 1H), 6.96 (s, 1H), 6.84-6.76 (m, 1H), 6.75-6.67 (m, 1H), 5.70-5.67 (m, 2H), 5.17 (t, J=5.7 Hz, 1H), 4.84 (d, J=4.6 Hz, 2H), 4.45 (d, J=5.8 Hz, 2H), 3.77 (s, 3H), 2.35-2.27 (m, 1H), 1.44 (s, 9H), 1.25-1.20 (m, 2H), 1.08-1.03 (m, 2H).

LC RT: 0.77 min. LC/MS [M+H]⁺=523.4 (Method D)

Step 2. A solution of tert-butyl (7-(((5-cyclopropyl-1,2,4-oxadiazol-3-yl)methyl)-amino)-1-(4-(hydroxymethyl)-2-methoxybenzyl)-1H-pyrazolo[4,3-d]pyrimidin-5-yl)carbamate (93.2 mg, 0.178 mmol) in DCM (3.6 mL) was treated with SOCl₂ (39 μL, 0.54 mmol). The reaction mixture was stirred at RT for 10 min and concentrated in vacuo. The residue was dissolved in DCM and concentrated in vacuo to give tert-butyl (1-(4-(chloromethyl)-2-methoxybenzyl)-7-(((5-cyclopropyl-1,2,4-oxadiazol-3-yl)methyl)amino)-1H-pyrazolo[4,3-d]pyrimidin-5-yl)carbamate (95.4 mg, 99% yield).

¹H NMR (400 MHz, DMSO-d₆) δ 11.70-11.19 (m, 1H), 9.46-9.20 (m, 1H), 8.10-8.06 (m, 1H), 7.10 (s, 1H), 6.97 (s, 2H), 5.79 (s, 2H), 4.97 (br d, J=5.2 Hz, 2H), 4.73 (s, 2H), 3.74 (s, 3H), 2.40-2.30 (m, 1H), 1.53 (s, 9H), 1.30-1.22 (m, 2H), 1.10-1.04 (m, 2H).

LC RT: 0.89 min. LC/MS [M+H]⁺=541.3 (Method D).

Step 3. A solution of tert-butyl (1-(4-(chloromethyl)-2-methoxybenzyl)-7-(((5-cyclopropyl-1,2,4-oxadiazol-3-yl)methyl)amino)-1H-pyrazolo[4,3-d]pyrimidin-5-yl)carbamate (30 mg, 0.055 mmol) in DMF (1.1 mL) was treated with DIEA (77 μL, 0.44 mmol) and tetrahydro-2H-pyran-4-amine (22.4 mg, 0.222 mmol). The reaction mixture was stirred at 60° C. for 1 h, after which the temperature was raised to 65° C. and stirring continued for 1 h. The reaction mixture was dried under a N₂ stream followed by further drying in vacuo. The residue was dissolved in dioxane (1.1 mL) and treated with HCl (4 M in dioxane, 0.75 mL, 3 mmol), stirred at 40° C. for 90 min and concentrated in vacuo. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 2% B, 2-42% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The isolated product was purified further via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-70% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 127 (13.6 mg, 47%). See Table A for analytical data.

Compound 128 and Compound 129 were analogously prepared.

### Example 14—Compound 130

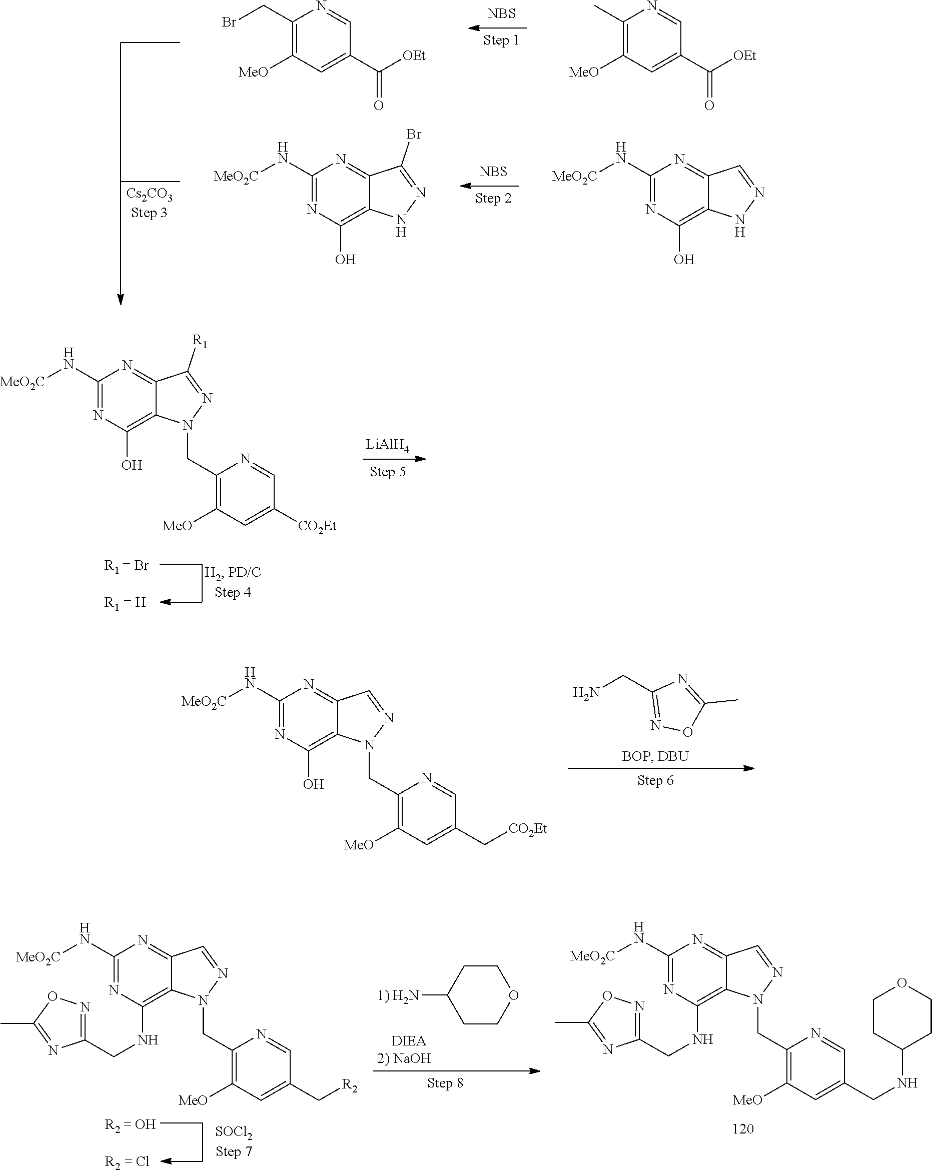

Step 1. A solution of ethyl 5-methoxy-6-methylnicotinate (1.32 g, 6.77 mmol) in CCl₄ (19 mL) was treated with NBS (1.44 g, 8.12 mmol) and AIBN (0.22 g, 1.4 mmol). The reaction mixture was stirred at 60° C. for 40 h and was washed with saturated aqueous Na₂S₂O₃ solution. The organic layer was concentrated in vacuo and the crude product was purified via column chromatography (40 g SiO₂; 0 to 25% EtOAc-Hexanes gradient elution) to give ethyl 6-(bromomethyl)-5-methoxynicotinate (1.20 g, 4.38 mmol, 65% yield).

¹H NMR (400 MHz, CHLOROFORM-d) δ 8.83-8.75 (m, 1H), 7.78 (d, J=1.6 Hz, 1H), 4.65 (s, 2H), 4.43 (q, J=7.1 Hz, 2H), 3.99 (s, 3H), 1.43 (t, J=7.2 Hz, 3H). LC RT: 0.89 min.

LC/MS [M+H]⁺=274.1 (Method D).

Step 2. A solution of methyl (7-hydroxy-1H-pyrazolo[4,3-d]pyrimidin-5-yl)carbamate (2.51 g, 12.0 mmol) in DMF (50 mL) was treated with NBS (2.14 g, 12.0 mmol). The reaction mixture was stirred at RT for 15 min and filtered. The collected solid was washed with H₂O and diethyl ether to give methyl (3-bromo-7-hydroxy-1H-pyrazolo[4,3-d]pyrimidin-5-yl)carbamate (3.28 g, 95% yield).

LC RT: 0.57 min. LC/MS [M+H]⁺=288.1 (Method D).

Step 3. A solution of methyl (3-bromo-7-hydroxy-1H-pyrazolo[4,3-d]pyrimidin-5-yl)carbamate (648 mg, 2.25 mmol) in DMF (22.5 mL) was treated with ethyl 6-(bromomethyl)-5-methoxynicotinate (617 mg, 2.25 mmol) and Cs₂CO₃ (2199 mg, 6.75 mmol). The reaction mixture was stirred at RT for 2 h, diluted with EtOAc, and washed with saturated NaHCO₃ solution and H₂O. The organic layer was concentrated in vacuo. The crude product was purified via column chromatography (40 g SiO₂; 0 to 100% EtOAc-Hexanes gradient elution) to give ethyl 6-((3-bromo-7-hydroxy-5-((methoxycarbonyl)amino)-1H-pyrazolo[4,3-d]pyrimidin-1-yl)methyl)-5-methoxynicotinate (653.1 mg, 60% yield).

¹H NMR (500 MHz, DMSO-d₆) δ 11.61-11.41 (m, 1H), 8.49-8.47 (m, 1H), 7.81 (d, J=1.6 Hz, 1H), 5.85 (s, 2H), 4.34 (q, J=7.1 Hz, 2H), 3.96 (s, 3H), 3.74 (s, 3H), 1.31 (t, J=7.1 Hz, 3H).

LC RT: 0.86 min. LC/MS [M+H]⁺=481.2 (Method D).

Step 4. A suspension of ethyl 6-((3-bromo-7-hydroxy-5-((methoxycarbonyl)amino)-1H-pyrazolo[4,3-d]pyrimidin-1-yl)methyl)-5-methoxynicotinate (542 mg, 1.13 mmol) in MeOH (54 mL) was treated with Pd/C (24 mg, 0.23 mmol). The reaction flask was evacuated under vacuum and purged with H₂ (3×). The reaction mixture was stirred under a H₂ atmosphere (balloon) for 16 h. The reaction flask was evacuated under vacuum and purged with N₂ (3×). The reaction mixture was diluted with DCM, filtered through CELITE™ and concentrated in vacuo give ethyl 6-((7-hydroxy-5-((methoxycarbonyl)amino)-1H-pyrazolo[4,3-d]pyrimidin-1-yl)methyl)-5-methoxynicotinate (450 mg, 99% yield).

¹H NMR (400 MHz, DMSO-d₆) δ 8.49-8.44 (m, 1H), 7.85 (s, 1H), 7.79 (d, J=1.6 Hz, 1H), 5.86 (s, 2H), 4.33 (q, J=7.1 Hz, 2H), 3.95 (s, 3H), 3.75 (s, 3H), 1.31 (t, J=7.1 Hz, 3H).

LC RT: 0.78 min. LC/MS [M+H]⁺=403.0 (Method D)

Step 5. A solution of ethyl 6-((7-hydroxy-5-((methoxycarbonyl)amino)-1H-pyrazolo[4,3-d]pyrimidin-1-yl)methyl)-5-methoxynicotinate (543 mg, 1.35 mmol) in THF (28 mL) was cooled to 0° C. and treated with LiAlH₄ (1 M in THF, 2.4 mL, 2.4 mmol). The reaction mixture was stirred for 15 min at 0° C., quenched with H₂O and Rochelle salt (saturated aqueous solution), and stirred at RT for 2 h. The organic layer was absorbed onto CELITE™ and purified via column chromatography (40 g SiO₂; 0 to 10% MeOH-DCM gradient elution) to give methyl (7-hydroxy-1-((5-(hydroxymethyl)-3-methoxypyridin-2-yl)methyl)-1H-pyrazolo[4,3-d]pyrimidin-5-yl)carbamate (191 mg, 39% yield).

¹H NMR (400 MHz, DMSO-d₆) δ 7.89-7.84 (m, 1H), 7.80 (s, 1H), 7.37 (d, J=1.5 Hz, 1H), 5.80-5.72 (m, 2H), 5.28 (t, J=5.7 Hz, 1H), 4.48 (d, J=5.4 Hz, 2H), 3.87-3.81 (m, 3H), 3.74 (s, 3H).

LC RT: 0.56 min. LC/MS [M+H]⁺=361.0 (Method D).

Step 6. A solution of methyl (7-hydroxy-1-((5-(hydroxymethyl)-3-methoxypyridin-2-yl)methyl)-1H-pyrazolo[4,3-d]pyrimidin-5-yl)carbamate (190 mg, 0.527 mmol) in DMSO (2.6 mL) was treated with (5-methyl-1,2,4-oxadiazol-3-yl)methanamine-HCl (103 mg, 0.685 mmol), BOP (303 mg, 0.685 mmol) and DBU (0.28 mL, 1.8 mmol). The reaction mixture was stirred at RT for 1 h, diluted with DCM, and washed with H₂O (6×). The organic layer was concentrated in vacuo. The crude product was dissolved in MeOH, filtered through a PTFE frit, and purified via preparative HPLC with the following conditions: Column: Axia C18 100 mm×30 mm, 5-μm particles; Mobile Phase A: 10:90 Methanol: water with 0.1% TFA; Mobile Phase B: 90:10 Methanol: water with 0.1% TFA; Gradient: a 0-minute hold at 5% B, 5-30% B over 10 minutes, then a 2-minute hold at 30% B; Flow Rate: 40 mL/min; UV detection at 220 nm; Column Temperature: 25° C. The purified product was neutralized with saturated aqueous NaHCO₃ solution and washed with DCM. The organic layer was dried over Na₂SO₄, filtered and concentrated in vacuo to give methyl (1-((5-(hydroxymethyl)-3-methoxypyridin-2-yl)methyl)-7-(((5-methyl-1,2,4-oxadiazol-3-yl)methyl)amino)-1H-pyrazolo[4,3-d]pyrimidin-5-yl)carbamate (102.4 mg, 43% yield).

¹H NMR (400 MHz, DMSO-d₆) δ 9.68 (s, 1H), 8.99 (br s, 1H), 7.98-7.92 (m, 1H), 7.84 (s, 1H), 7.45 (d, J=1.1 Hz, 1H), 5.77 (s, 2H), 5.35 (br s, 1H), 4.92 (br s, 2H), 4.51 (br s, 2H), 3.88 (s, 3H), 3.61 (s, 3H), 2.57 (s, 3H).

LC RT: 0.61 min. LC/MS [M+H]⁺=456.1 (Method D).

Step 7. A solution of methyl (1-((5-(hydroxymethyl)-3-methoxypyridin-2-yl)methyl)-7-(((5-methyl-1,2,4-oxadiazol-3-yl)methyl)amino)-1H-pyrazolo[4,3-d]pyrimidin-5-yl)carbamate (102 mg, 0.225 mmol) in DCM (4.5 mL) was treated with SOCl₂ (49 μL, 0.68 mmol). The reaction mixture was stirred at RT for 30 min and concentrated in vacuo. The residue was dissolved in DCM and concentrated in vacuo to give methyl (1-((5-(chloromethyl)-3-methoxypyridin-2-yl)methyl)-7-(((5-methyl-1,2,4-oxadiazol-3-yl)methyl)amino)-1H-pyrazolo[4,3-d]pyrimidin-5-yl)carbamate (107 mg, 100% yield).

LC RT: 0.67 min. LC/MS [M+H]⁺=474.3 (Method D).

Step 8. A solution of methyl (1-((5-(chloromethyl)-3-methoxypyridin-2-yl)methyl)-7-(((5-methyl-1,2,4-oxadiazol-3-yl)methyl)amino)-1H-pyrazolo[4,3-d]pyrimidin-5-yl)carbamate (35 mg, 0.074 mmol) in DMF (0.7 mL) was treated with DIEA (103 μL, 0.591 mmol) and tetrahydro-2H-pyran-4-amine (29.9 mg, 0.295 mmol). The reaction mixture was stirred at 70° C. for 2 h and dried under a N₂ stream followed by further drying in vacuo. The residue was dissolved in dioxane (0.8 mL) and treated with NaOH (10M aqueous solution, 37 μL, 0.37 mmol). The reaction mixture was heated to 60° C. Additional NaOH (10M aqueous solution, 120 μL, 1.2 mmol) were added to the reaction mixture over a period of 8 h. The reaction mixture was neutralized at RT with HOAc and concentrated in vacuo. The crude product was dissolved in DMF, filtered through a PTFE frit, and purified via preparative LC/MS with the following conditions: Column: XBridge C18, 200 mm×19 mm, 5-μm particles; Mobile Phase A: 5:95 acetonitrile: water with 10 mM NH₄OAc; Mobile Phase B: 95:5 acetonitrile: water with 10 mM NH₄OAc; Gradient: a 0-minute hold at 1% B, 1-41% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The isolated product was purified further via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 0% B, 0-40% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 130 as the bis TFA salt (11 mg, 20%).

Compound 131 was analogously prepared.

### Example 15—Compound 134

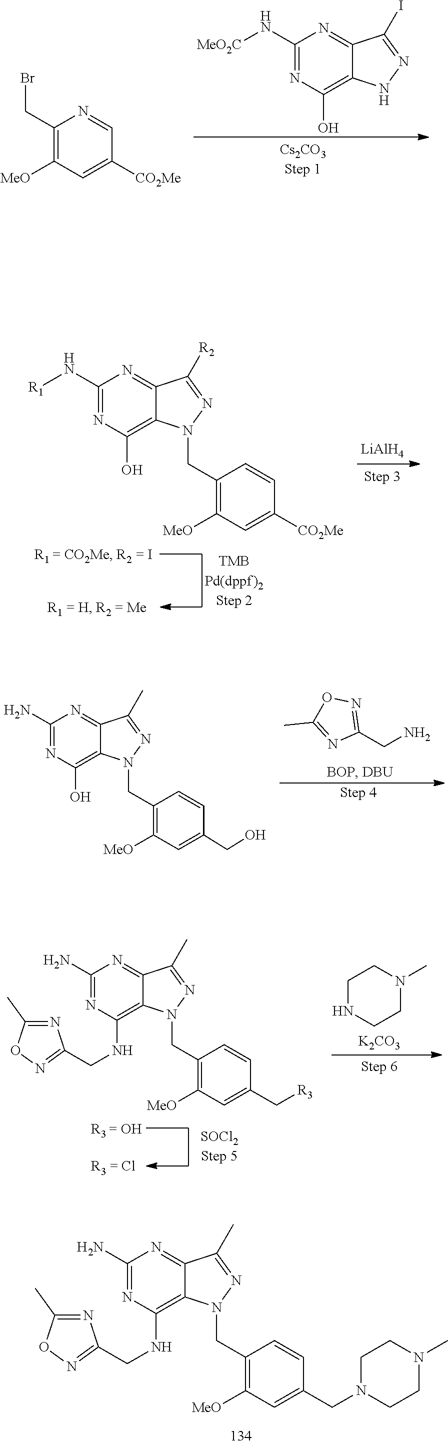

Step 1. To a stirred solution of methyl (7-hydroxy-3-iodo-1H-pyrazolo[4,3-d]pyrimidin-5-yl)carbamate (5.0 g, 14.92 mmol) in DMF (50.0 mL) at 0° C., were added Cs₂CO₃ (9.72 g, 29.8 mmol) and methyl 4-(bromomethyl)-3-methoxybenzoate (3.87 g, 14.92 mmol). The reaction mixture was stirred at 0° C. for 1 h and water was added. The precipitated solid was filtered and washed with excess of water followed by petroleum ether. The solid was dried under vacuum. The crude compound was purified by ISCO combiflash chromatography by eluting with 0-100% ethyl acetate in chloroform to afford methyl 4-((7-hydroxy-3-iodo-5-((methoxycarbonyl)amino)-1H-pyrazolo[4,3-d]pyrimidin-1-yl)methyl)-3-methoxybenzoate (3.88 g, 6.20 mmol, 41.5% yield) as an off-white solid.

¹H NMR (400 MHz, DMSO-d₆) δ ppm: 11.69 (br s, 1H), 11.38 (s, 1H), 7.56-7.45 (m, 2H), 6.87-6.78 (m, 1H), 5.75 (s, 2H), 3.88 (s, 3H), 3.85 (s, 3H), 3.75 (s, 3H).

LC-MS m/z 514.0 [M+H]⁺.

Step 2. To a stirred solution of methyl 4-((7-hydroxy-3-iodo-5-((methoxycarbonyl)-amino)-1H-pyrazolo[4,3-d]pyrimidin-1-yl)methyl)-3-methoxybenzoate (3.5 g, 6.82 mmol) in 1,4-dioxane (35.0 mL), were added K₂CO₃ (1.885 g, 13.64 mmol), TMB (1.907 mL, 13.64 mmol) and PdCl₂(dppf).CH₂Cl₂ adduct (0.557 g, 0.682 mmol) under N₂ purging. The reaction mixture was stirred at 100° C. for 6 h. The reaction mixture was filtered through CELITE™ bed and washed with excess of ethyl acetate. The filtrate was concentrated under reduced pressure to afford the residue. The crude compound was purified by ISCO combiflash chromatography (0-20% methanol in chloroform) to afford methyl 4-((5-amino-7-hydroxy-3-methyl-1H-pyrazolo[4,3-d]pyrimidin-1-yl)methyl)-3-methoxybenzoate (2.1 g, 4.10 mmol, 60.1% yield) as a brown solid.

¹H NMR (400 MHz, DMSO-d₆) δ=10.90 (s, 1H), 7.51 (s, 1H), 7.46 (d, J=8.0 Hz, 1H), 6.63-6.50 (m, 1H), 6.18-6.01 (m, 2H), 5.71-5.54 (m, 2H), 3.91 (s, 3H), 3.87-3.78 (s, 3H), 2.23 (s, 3H).

LC-MS m/z 344.0 [M+H]⁺.

Step 3. To a stirred solution of methyl 4-((5-amino-7-hydroxy-3-methyl-1H-pyrazolo[4,3-d]pyrimidin-1-yl)methyl)-3-methoxybenzoate (0.5 g, 1.456 mmol) in THF (5.0 mL) at 0° C., was added LiAlH₄ (1.214 mL, 2.91 mmol). The reaction mixture was warmed to RT, stirred for 1 h, quenched with ice cold water and filtered through a CELITE™ bed, which was washed with excess of ethyl acetate. The organic layer was dried over Na₂SO₄, filtered and concentrated under reduced pressure to afford 5-amino-1-(4-(hydroxymethyl)-2-methoxybenzyl)-3-methyl-1H-pyrazolo[4,3-d]pyrimidin-7-ol (0.31 g, 0.551 mmol, 37.8% yield) as a brown semi-solid.

¹H NMR (400 MHz, DMSO-d₆) δ=6.99-6.95 (m, 1H), 6.73 (br d, J=7.5 Hz, 1H), 6.44-6.38 (m, 1H), 5.75-5.49 (m, 2H), 5.26-4.99 (m, 1H), 4.44 (s, 2H), 3.87-3.80 (m, 3H), 2.23 (s, 3H).

LC-MS m/z 316.3 [M+H]⁺.

Step 4. To a stirred solution of 5-amino-1-(4-(hydroxymethyl)-2-methoxybenzyl)-3-methyl-1H-pyrazolo[4,3-d]pyrimidin-7-ol (1.1 g, 3.49 mmol) in DMSO (10.0 mL), were added DBU (1.577 mL, 10.47 mmol), BOP (2.314 g, 5.23 mmol) and (5-methyl-1,2,4-oxadiazol-3-yl)methanamine hydrochloride (0.522 g, 3.49 mmol). The reaction mixture was stirred at RT for 2 h. (5-Methyl-1,2,4-oxadiazol-3-yl)methanamine, HCl (0.3 g, 2.0 mmol) was added. The reaction mixture was stirred at RT for 16 h and partitioned between EtOAc and water. The organic layer was washed with brine, dried over Na₂SO₄, filtered and concentrated under reduced pressure to afford the residue. The crude compound was purified by ISCO combiflash chromatography by eluting with 0-20% methanol in chloroform to afford (4-((5-amino-3-methyl-7-(((5-methyl-1,2,4-oxadiazol-3-yl)methyl)amino)-1H-pyrazolo[4,3-d]pyrimidin-1-yl)methyl)-3-methoxyphenyl)methanol (0.81 g, 1.243 mmol, 35.6% yield) as a brown solid.

¹H NMR (400 MHz, DMSO-d₆) δ=7.60-7.55 (m, 1H), 7.26 (br t, J=5.8 Hz, 1H), 6.98-6.93 (m, 1H), 6.77 (br d, J=7.5 Hz, 1H), 6.68-6.60 (m, 1H), 5.68 (s, 2H), 5.55-5.48 (m, 1H), 5.20-5.13 (m, 1H), 4.78 (br d, J=5.5 Hz, 2H), 4.49-4.42 (m, 2H), 3.82-3.77 (m, 3H), 2.56 (d, J=2.0 Hz, 4H), 2.55-2.50 (m, 6H).

LC-MS m/z 411.2 [M+H]⁺.

Step 5. To a stirred solution of (4-((5-amino-3-methyl-7-(((5-methyl-1,2,4-oxadiazol-3-yl)methyl)amino)-1H-pyrazolo[4,3-d]pyrimidin-1-yl)methyl)-3-methoxyphenyl)methanol (0.45 g, 1.096 mmol) in THF (10.0 mL) at 0° C., was added SOCl₂ (1.0 ml, 13.70 mmol). The reaction mixture was stirred at 0° C. for 1 h, warmed to RT, and concentrated under reduced pressure to afford crude 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.51 g, assumed 100% yield) as a brown solid, which was used as such in the next step.

LC-MS m/z 429.4 [M+H]⁺.

Step 6. To a stirred solution of 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.15 g, 0.350 mmol) in DMF (3.0 mL), were added 1-methylpiperazine (0.053 g, 0.525 mmol) and K₂CO₃ (0.145 g, 1.049 mmol). The reaction mixture was stirred at 50° C. for 90 min and filtered through a CELITE™ bed, which was washed with excess ethyl acetate. The filtrate was concentrated under reduced pressure to afford the residue. The crude compound was purified by reversed phase preparative LC/MS (Column: TRIART-YMC-EXRS (250 mm×19 mm); mobile phase A: 10 mM NH₄OAc in water pH-4.5, mobile phase B: CH₃CN; flow rate: 20 mL/min; gradient: 0/0, 10/15, 20/15, 22/100, 24/0). The fraction collection was triggered by MS and UV signals. The fractions containing the desired product were combined and dried via centrifugal evaporation using a Genevac apparatus to afford Compound 134 (12.6 mg, 0.025 mmol, 7.15% yield).

### Example 16—Compound 132

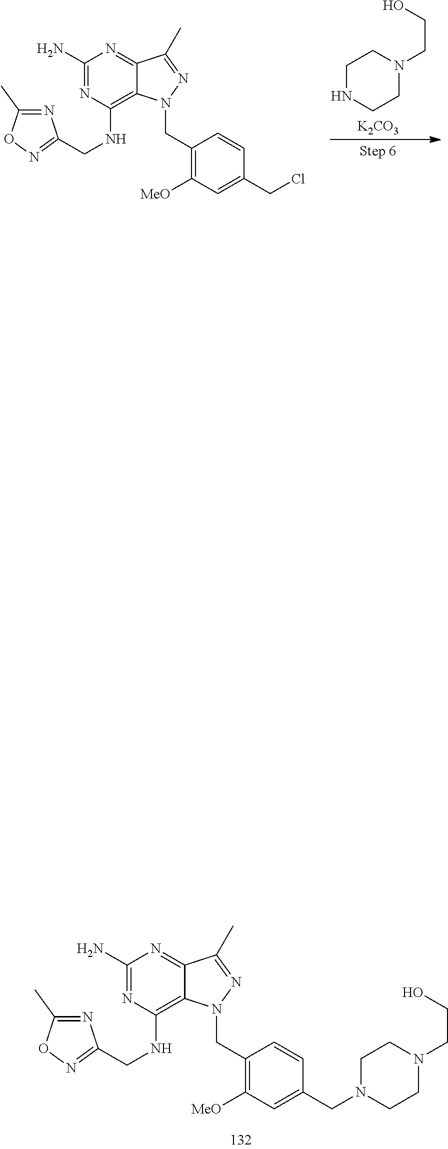 [SMILES: COc1cc(CCl)ccc1Cn1nc(C)c2nc(N)nc(NCc3noc(C)n3)c21 ; "K2CO3" ; OCCN1CCNCC1 ; COc1cc(CN2CCN(CCO)CC2)ccc1Cn1nc(C)c2nc(N)nc(NCc3noc(C)n3)c21]

To a stirred solution of 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.15 g, 0.350 mmol) in DMF (3.0 mL), were added 2-(piperazin-1-yl)ethan-1-ol (0.068 g, 0.525 mmol), 2-(piperazin-1-yl)ethan-1-ol (0.068 g, 0.525 mmol) and K₂CO₃ (0.097 g, 0.699 mmol). The reaction mixture was stirred at 50° C. for 90 min and filtered through a CELITE™ bed, which washed with excess ethyl acetate. The filtrate was concentrated under reduced pressure to afford a residue, which was purified by reversed phase preparative LC/MS (column: Gemini NX (250×21.2 mm) 5 μm, mobile phase A: 10 mM ammonium bicarbonate in water 9.5 pH, mobile phase B: CH₃CN, flow rate: 20 mL/min, gradient T/% B: 0/10, 7/35, 12/35, 12.01/100). Fraction collection was triggered by MS and UV signals. Fractions containing the desired product were combined and dried via centrifugal evaporation using a Genevac apparatus to afford Compound 132 (51.2 mg, 0.095 mmol, 27.2% yield).

### Example 17—Compound 133

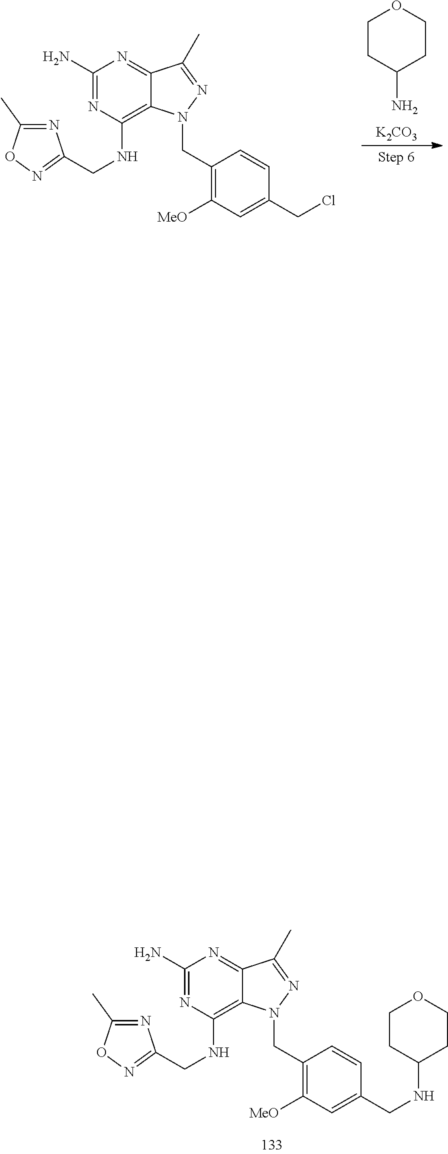 [SMILES: COc1cc(CCl)ccc1Cn1nc(C)c2nc(N)nc(NCc3noc(C)n3)c21 ; "K2CO3" ; NC1CCOCC1 ; COc1cc(CNC2CCOCC2)ccc1Cn1nc(C)c2nc(N)nc(NCc3noc(C)n3)c21]

To a stirred solution of 1-(4-(chloromethyl)-2-methoxybenzyl)-3-methyl-N7-((5-methyl-1,2,4-oxadiazol-3-yl)methyl)-1H-pyrazolo[4,3-d]pyrimidine-5,7-diamine (0.15 g, 0.350 mmol) in acetonitrile (3.0 mL), were added tetrahydro-2H-pyran-4-amine hydrochloride (0.072 g, 0.525 mmol), Na₂CO₃ (0.111 g, 1.049 mmol) and KI (0.058 g, 0.350 mmol). The reaction mixture was stirred at 50° C. for 3 h. The reaction mixture was filtered through a CELITE™ bed, which was washed with excess of ethyl acetate. The filtrate was concentrated under reduced pressure to afford the residue. The crude compound was purified by reversed phase preparative LC/MS (column: Gemini NX (250×21 mm)×5 micron; mobile phase A: 10 mM NH4OAc in water, mobile phase B: CH₃CN:MeOH (1:1), flow rate: 19 mL/min, gradient: 0/35, 12/45). The fraction collection was triggered by MS and UV signals. Fractions containing the desired product were combined and dried via centrifugal evaporation using Genevac to afford Compound 133 (17.4 mg, 0.035 mmol, 10.08% yield).

### Example 18—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

### Chart 1

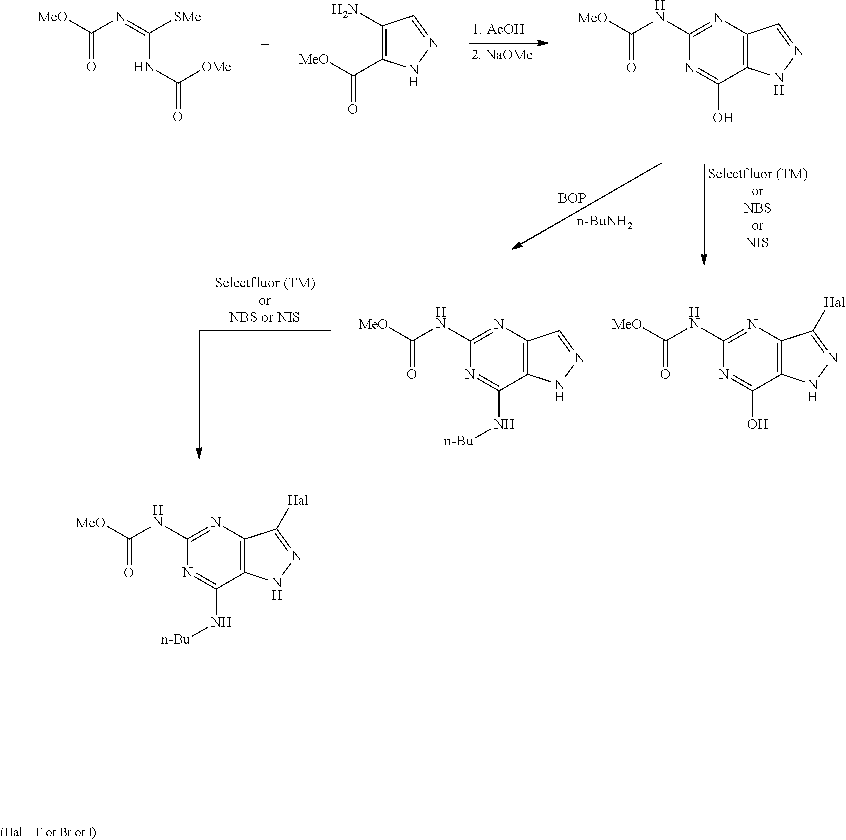

### Chart 2

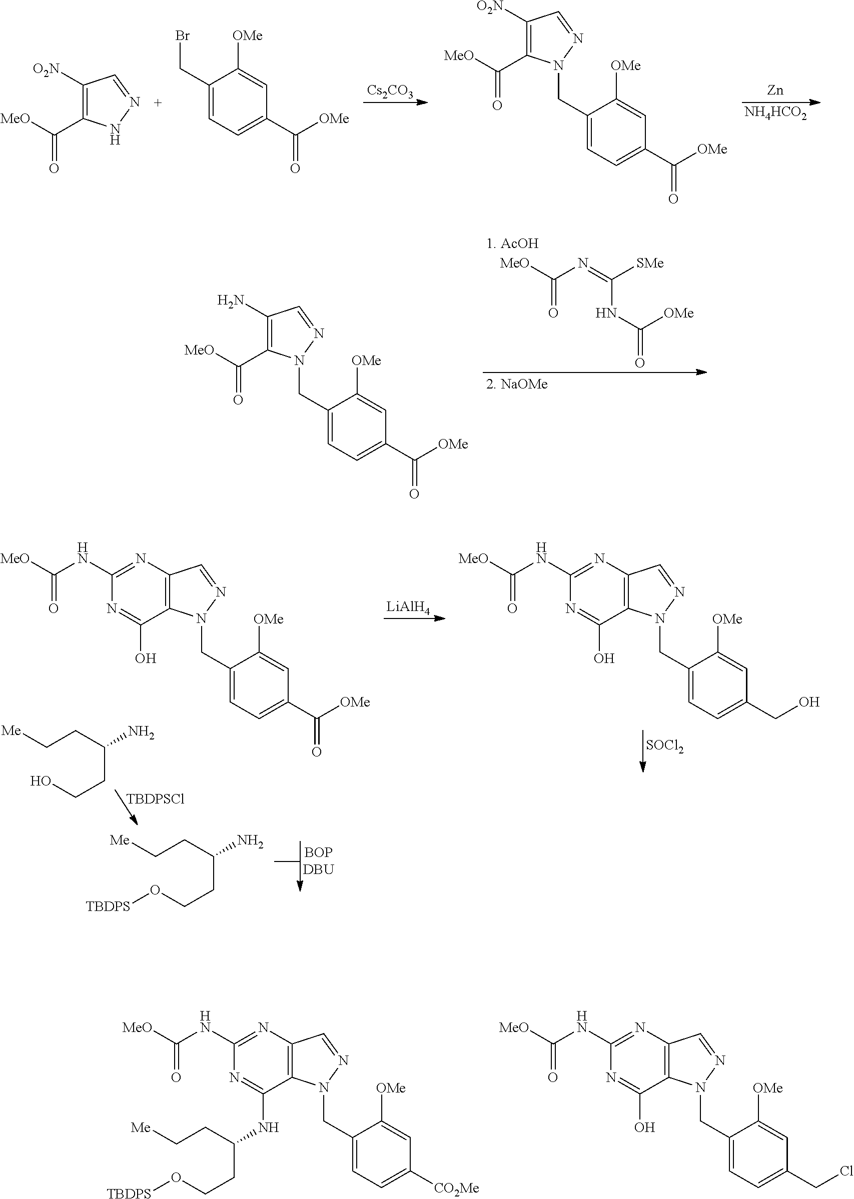

### Chart 3

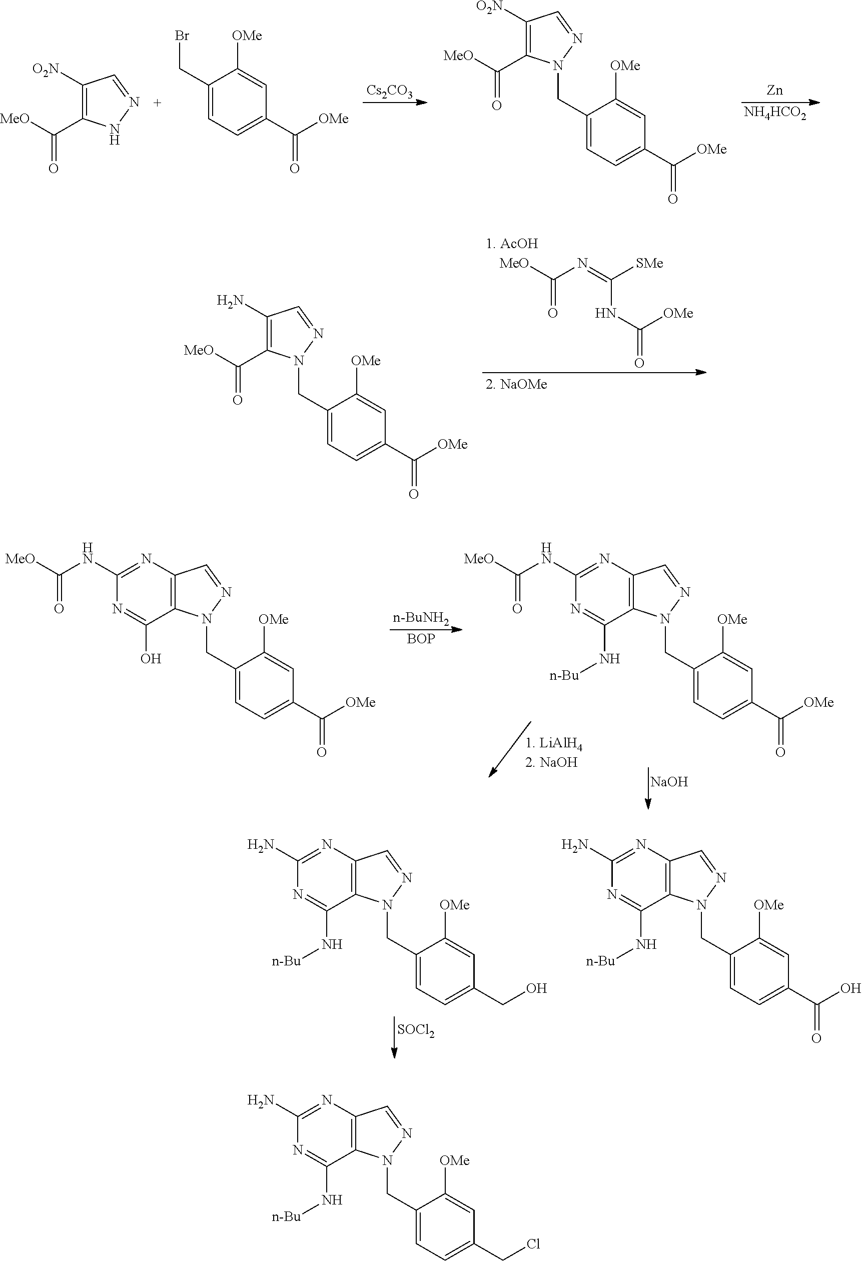

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×→1× in H₂O, warm at 37° C.; Cat # BD 558049) and kept the perm buffer (on ice) for later use.

For surface markers staining (CD69): prepared surface Abs: 0.045 ul hCD14-FITC (ThermoFisher Cat # MHCD1401)+0.6 ul hCD19-ef450 (ThermoFisher Cat #48-0198-42)+1.5 ul hCD69-PE (cat # BD555531)+0.855 ul FACS buffer. Added 3 ul/well, spin1000 rpm for 1 min and mixed on shaker for 30 sec, put on ice for 30 mins. Stop stimulation after 30 minutes with 70 uL of prewarmed 1× fix/lysis buffer and use Feliex mate to resuspend (15 times, change tips for each plate) and incubate at 37 C for 10 minutes.

Centrifuge at 2000 rpm for 5 minutes aspirate with HCS plate washer, mix on shaker for 30 sec and then wash with 70 uL in dPBS and pelleted 2×s (2000 rpm for 5 min) and 50 ul wash in FACS buffer pelleted 1×s(2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2× (spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hlgG+0.04 ul MX-1. Spin 1000 rpm for 1 min, mix on shaker for 30se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat # K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

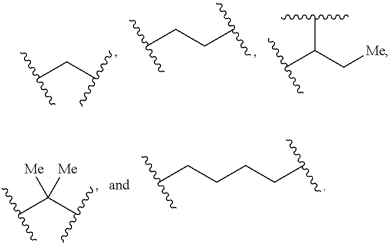

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cycloalkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (or “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₈ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

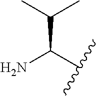 [SMILES: *[C@@H](N)C(C)C]

or that R is

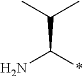 [SMILES: *[C@@H](N)C(C)C]

in the formula

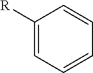

means

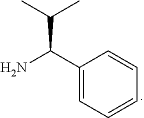

In the formulae of this specification, a bond traversing an aromatic ring between two carbons thereof means that the group attached to the bond may be located at any of the positions of the aromatic ring made available by removal of the hydrogen that is implicitly there (or explicitly there, if written out). By way of illustration:

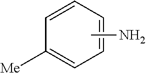

represents

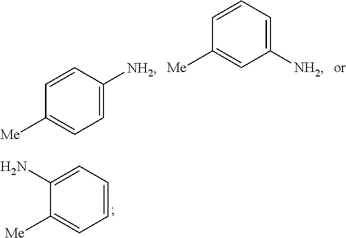

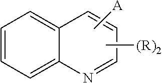

represents

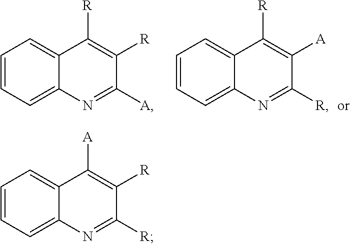

and

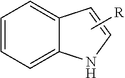

represents

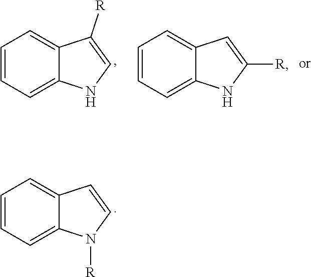

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

Table C provides a list of acronyms and abbreviations used in this specification, along with their meanings.

TABLE C

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5-b]pyridinium 3- |
| | oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, | Liquid chromatography-mass spectrometry |
| LC/MS | |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context | Retention time, in min |
| of liquid | |
| chromatography) | |
| RT (in the context | Room (ambient) temperature, circa 25° C. |
| of reaction | |
| conditions) | |
| Sat. | Saturated |
| Soln | Solution |
| TBDPS | tert-Butyldiphenylsilyl |
| TBS | t-Butyldimethylsilyl group |
| TEA | Triethylamine |
| TEAA | Triethylammonium acetate |
| TFA | TFA |
| THF | Tetrahydrofuran |
| TMB | 2,4,6-Trimethyl-1,3,5,2,4,6-trioxatriborinane; |
| | trimethylboroxine |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 A1 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039986 A1 (2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I or formula (II) 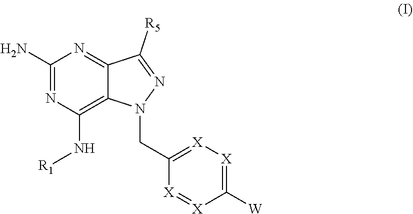
wherein
W is H, halo, C₁-C₃ alkyl, CN, (C₁-C₄ alkanediyl)OH, 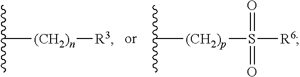
each X is independently N or CR²;
R¹ is (C₁-C₈ alkanediyl)₀₋₁(C₃ cycloalkyl),
(C₁-C₈ alkanediyl)₀₋₁(C₅-C₆ cycloalkyl),
(C₁-C₄ alkanediyl)₀₋₁(5-6 membered heteroaryl),
(C₁-C₄ alkanediyl)₀₋₁phenyl, or
(C₁-C₄ alkanediyl)CF₃;
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R³ is H, halo, OH, CN,
NH₂,
NH[C(═O)]₀₋₁(C₁-C₅ alkyl),
N(C₁-C₅ alkyl)₂,
NH[C(═O)]₀₋₁(C₁-C₄ alkanediyl)₀₋₁(C₃-C₅ cycloalkyl),
N(C₃-C₆ cycloalkyl)₂,
N[C₁-C₃ alkyl]C(═O)(C₁-C₆ alkyl),
NH(SO₂)(C₁-C₅ alkyl),
NH(SO₂)(C₁-C₄ alkanediyl)₀₋₁(C₃-C₅ cycloalkyl),
a 6-membered aromatic or heteroaromatic moiety,
a 5-membered heteroaromatic moiety, or
a moiety having the structure 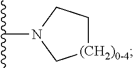
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 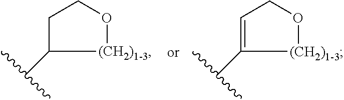
R⁶ is NH₂,
(NH)₀₋₁(C₁-C₅ alkyl),
N(C₁-C₅ alkyl)₂,
(NH)₀₋₁(C₁-C₄ alkanediyl)₀₋₁(C₃-C₅ cycloalkyl),
N(C₃-C₆ cycloalkyl)₂,
or
a moiety having the structure 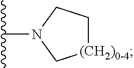
R^{x} and R^{y} are independently H or C₁-C₃ alkyl or R^{x} and R^{y} combine with the nitrogen to which they are bonded to form a 3- to 7-membered ring
n is 1, 2, or 3;
and
p is 0, 1, 2, or 3;
wherein in R¹, R², R³, and R⁵
an alkyl moiety, alkanediyl moiety, cycloalkyl moiety, or moiety of the formula 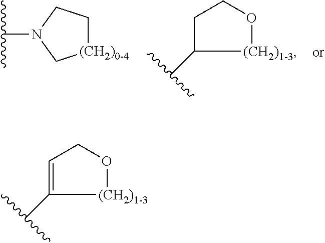
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl); and
an alkyl, alkanediyl, cycloalkyl, or moiety of the formula 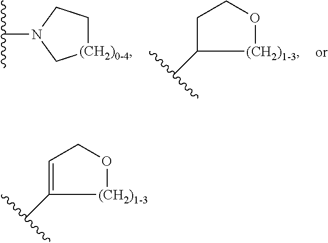
may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

2. A compound according to claim 1, wherein R¹ is selected from the group consisting of

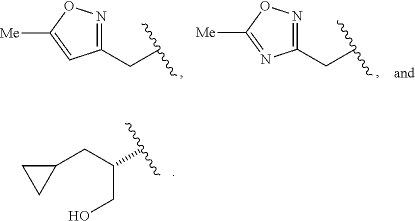 [SMILES: *Cc1cc(C)on1 ; *Cc1noc(C)n1 ; *[C@H](CO)CC1CC1]

3. A compound according to claim 1, wherein R² is OMe.

4. A compound according to claim 1, wherein R³ is selected from the group consisting of

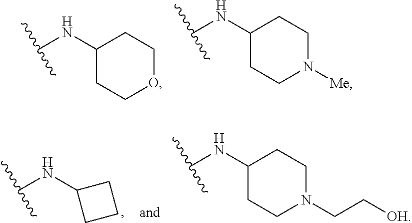

5. A compound according to claim 1, wherein R⁵ is H.

6. A compound according to claim 1, having a structure according to formula (Ia)

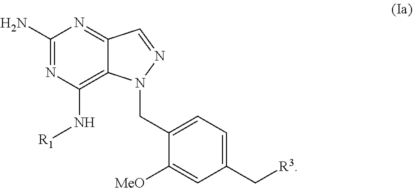

7. A compound according to claim 6, wherein R¹ is selected from the group consisting of

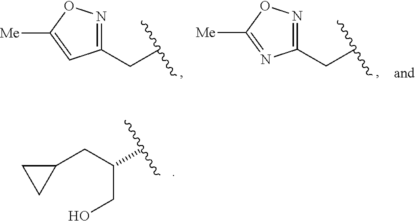 [SMILES: *Cc1cc(C)on1 ; *Cc1noc(C)n1 ; *[C@H](CO)CC1CC1]

8. A compound according to claim 6, wherein R³ is selected from the group consisting of

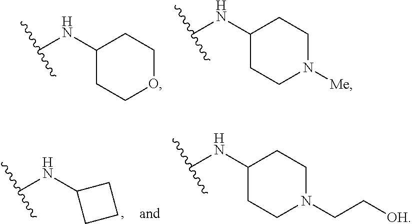

9. A compound having a structure according to formula (Ia) 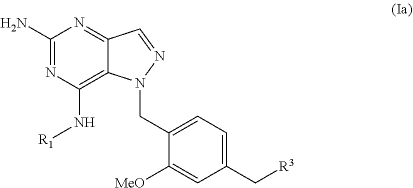
wherein
R¹ is 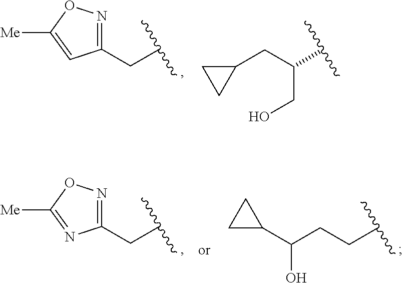
and
R³ is OH,

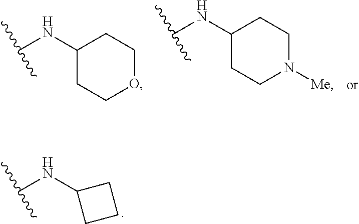

10. A method of treating a cancer, comprising administering to a patient suffering from such cancer a therapeutically effective combination of an anti-cancer immunotherapy agent and a compound according to claim 1.

11. A method according to claim 10, wherein the anti-cancer immunotherapy agent is an antagonistic anti-CTLA-4, anti-PD-1, or anti-PD-L¹ antibody.

12. A method according to claim 11, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

13. A method according to claim 12, wherein the anti-cancer immunotherapy agent is ipilimumab, nivolumab, or pembrolizumab.

14. A method of treating a cancer, comprising administering to a patient suffering from such cancer a therapeutically effective combination of an anti-cancer immunotherapy agent and a compound according to claim 9.

15. A method according to claim 14, wherein the anti-cancer immunotherapy agent is an antagonistic anti-CTLA-4, anti-PD-1, or anti-PD-L¹ antibody.

16. A method according to claim 14, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

17. A method according to claim 16, wherein the anti-cancer immunotherapy agent is ipilimumab, nivolumab, or pembrolizumab.
